SEQUENCE LISTING (23 sequences)

SEQ ID NO: 1 - protein, 168 aa, Artificial Sequence
  Artificial Sequence derived from Homo sapiens sequence
HGEGTFTSDVSSYLEGQAAKEFIAWLVKGRHGEGTFTSDVSSYLEGQAAKEFIAWLVKGRDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKA

SEQ ID NO: 2 - protein, 163 aa, Artificial Sequence
  Artificial Sequence derived from Homo sapiens sequence
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 3 - protein, 148 aa, Artificial Sequence
  Artificial Sequence derived from Homo sapiens sequence
HGEGTFTSDLSKQMEEEAVRLFIEWLKNGGPSSGAPPPSGDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 4 - protein, 188 aa, Artificial Sequence
  Artificial Sequence derived from Homo sapiens sequence
HGEGTFTSDLSKQMEEEAVRLFIEWLKNGGPSSGAPPPSGKEAAAKEAAAKEAAAKELAAKEAAAKEAAAKEAAAKELAADIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 5 - protein, 153 aa, Artificial Sequence
  Artificial Sequence derived from Homo sapiens sequence
HGEGTFTSDVSSYLEGQAAKEFIAWLVKGRGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 6 - protein, 142 aa, Artificial Sequence
  Artificial Sequence derived from Homo sapiens sequence
HGEGTFTSDVSSYLEGQAAKEFIAWLVKGRGPSSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 7 - protein, 179 aa, Artificial Sequence
  Artificial Sequence derived from Homo sapiens sequence
HGEGTFTSDVSSYLEGQAAKEFIAWLVKGRGKEAAAKEAAAKEAAAKELAAKEAAAKEAAAKEAAAKELAADIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 8 - protein, 108 aa, Artificial Sequence
  Artificial Sequence derived from Homo sapiens sequence
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 9 - protein, 31 aa, Artificial Sequence
  Artificial Sequence derived from Homo sapiens sequence
HGEGTFTSDVSSYLEGQAAKEFIAWLVKGRG

SEQ ID NO: 10 - protein, 39 aa, Heloderma suspectum
HGEGTFTSDLSKQMEEEAVRLFIEWLKNGGPSSGAPPPS

SEQ ID NO: 11 - protein, 40 aa, Artificial Sequence
  Artificial Sequence derived from Homo sapiens sequence
KEAAAKEAAAKEAAAKELAAKEAAAKEAAAKEAAAKELAA

SEQ ID NO: 12 - protein, 16 aa, Artificial Sequence
  Artificial Sequence derived from Homo sapiens sequence
GGGGGSGGGGSGGGGS

SEQ ID NO: 13 - DNA, 504 bp, Artificial Sequence
  Artificial Sequence derived from Homo sapiens sequence
catggtgaagggacctttaccagtgatgtaagttcttatttggaaggccaagctgccaaggaattcattgcttggctggtgaaaggccgacatggtgaagggacctttaccagtgatgtaagttcttatttggaaggccaagctgccaaggaattcattgcttggctggtgaaaggccgagacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 14 - DNA, 489 bp, Artificial Sequence
  Artificial Sequence derived from Homo sapiens sequence
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 15 - DNA, 495 bp, Artificial Sequence
  Artificial Sequence derived from Homo sapiens sequence
cacggtgaaggtaccttcacctctgacctgagcaaacagatggaggaagaagcggttcgtctgttcatcgagtggctgaaaaacggtggtccgtcttctggtgctccgccaccgtctggtggtggtggtggttctggtggtggtggttctggtggtggcggtagcgacatccagatgactcagtccccaagctctctgtctgcctccgttggcgatcgtgttacgatcacgtgccgtgcttctcagtggatcggttcccagctgtcctggtatcagcagaaaccgggcaaagccccgaaactcctgatcatgtggcgtagctctctgcagtctggtgtaccgagccgcttctctggttctggttctggtaccgacttcaccctgaccatttcctctctgcagccggaagatttcgcgacctactactgtgctcagggtgcggcactgccacgtacttttggccagggtacgaaagtcgagattaaacgttaatga

SEQ ID NO: 16 - DNA, 444 bp, Artificial Sequence
  Artificial Sequence derived from Homo sapiens sequence
catggtgaaggaacatttaccagtgacttgtcaaaacagatggaagaggaggcagtgcggttatttattgagtggcttaagaacggaggaccaagtagcggggcacctccgccatcgggtgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 17 - DNA, 447 bp, Artificial Sequence
  Artificial Sequence derived from Homo sapiens sequence
cacggtgaaggtaccttcacctctgacctgagcaaacagatggaggaagaagcggttcgtctgttcatcgagtggctgaaaaacggtggtccgtcttctggtgctccgccaccgtctgacatccagatgactcagtccccaagctctctgtctgcctccgttggcgatcgtgttacgatcacgtgccgtgcttctcagtggatcggttcccagctgtcctggtatcagcagaaaccgggcaaagccccgaaactcctgatcatgtggcgtagctctctgcagtctggtgtaccgagccgcttctctggttctggttctggtaccgacttcaccctgaccatttcctctctgcagccggaagatttcgcgacctactactgtgctcagggtgcggcactgccacgtacttttggccagggtacgaaagtcgagattaaacgttaatga

SEQ ID NO: 18 - DNA, 564 bp, Artificial Sequence
  Artificial Sequence derived from Homo sapiens sequence
catggtgaaggaacatttaccagtgacttgtcaaaacagatggaagaggaggcagtgcggttatttattgagtggcttaagaacggaggaccaagtagcggggcacctccgccatcgggtaaagaagcggcggcgaaagaagcggcggcgaaagaagcggcggcgaaagaattggccgcaaaagaagcggcggcgaaagaagcggcggcgaaagaagcggcggcgaaagaattggccgcagacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 19 - DNA, 567 bp, Artificial Sequence
  Artificial Sequence derived from Homo sapiens sequence
cacggtgaaggtaccttcacctctgacctgagcaaacagatggaggaagaagcggttcgtctgttcatcgagtggctgaaaaacggtggtccgtcttctggtgctccgccaccgtctaaagaagcggcggcgaaagaagcggcggcgaaagaagcggcggcgaaagaattggccgcaaaagaagcggcggcgaaagaagcggcggcgaaagaagcggcggcgaaagaattggccgcagacatccagatgactcagtccccaagctctctgtctgcctccgttggcgatcgtgttacgatcacgtgccgtgcttctcagtggatcggttcccagctgtcctggtatcagcagaaaccgggcaaagccccgaaactcctgatcatgtggcgtagctctctgcagtctggtgtaccgagccgcttctctggttctggttctggtaccgacttcaccctgaccatttcctctctgcagccggaagatttcgcgacctactactgtgctcagggtgcggcactgccacgtacttttggccagggtacgaaagtcgagattaaacgttaatga

SEQ ID NO: 20 - DNA, 459 bp, Artificial Sequence
  Artificial Sequence derived from Homo sapiens sequence
catggtgaagggacctttaccagtgatgtaagttcttatttggaaggccaagctgccaaggaattcattgcttggctggtgaaaggccgaggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 21 - DNA, 426 bp, Artificial Sequence
  Artificial Sequence derived from Homo sapiens sequence
catggtgaagggacctttaccagtgatgtaagttcttatttggaaggccaagctgccaaggaattcattgcttggctggtgaaaggccgaggaccaagctcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 22 - DNA, 537 bp, Artificial Sequence
  Artificial Sequence derived from Homo sapiens sequence
catggtgaagggacctttaccagtgatgtaagttcttatttggaaggccaagctgccaaggaattcattgcttggctggtgaaaggccgaggaaaagaagcggcggcgaaagaagcggcggcgaaagaagcggcggcgaaagaattggccgcaaaagaagcggcggcgaaagaagcggcggcgaaagaagcggcggcgaaagaattggccgcagacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 23 - DNA, 324 bp, Artificial Sequence
  Artificial Sequence derived from Homo sapiens sequence
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

(19) United States
(12) Patent Application Publication
(10) Pub. No.: US 2013/0189255 A1
(43) Pub. Date: Jul. 25, 2013

(54) FUSIONS AND CONJUGATES OF INSULINOTROPIC AGENTS

(71) Applicant: GLAXO GROUP LIMITED, Greenford (GB)

(72) Inventors: Christopher Herring, Cambridge (GB); Lucy J. Holt, Cambridge (GB); Laurent Jespers, Cambridge (GB); Bruce Hamilton, Cambridge (GB)

(73) Assignee: GLAXO GROUP LIMITED, Greenford (GB)

(21) Appl. No.: 13/743,777

(22) Filed: Jan. 17, 2013

Related U.S. Application Data
(63) Continuation of application No. 12/935,591, filed on Sep. 30, 2010, now abandoned.
(60) Provisional application No. 61/040,796, filed on Mar. 31, 2008.
(60) Provisional application No. 61/086,891, filed on Aug. 7, 2008.

(51) Int. Cl. C07K 16/18; C07K 14/435; A61K 45/06; A61K 39/395

(52) U.S. Cl. CPC C07K 16/18; A61K 39/3955; C07K 14/435; A61K 45/06
USPC 424/134.1; 435/69.6; 435/328; 435/320.1; 530/387.3; 536/23.2

(57) ABSTRACT

The present invention relates to drug fusions of insulinotropic agents and incretin drugs that have improved serum half lives. These fusions and conjugates comprise polypeptides, immunoglobulin (antibody) single variable domains and GLP and/or exendin molecules. The invention further relates to uses, formulations, compositions and devices comprising such drug fusions and conjugates.

### CROSS-REFERENCE TO RELATED APPLICATIONS

This application is a continuation of U.S. patent application Ser. No. 12/935,591 filed Sep. 30, 2010; which is a 371 of International Application No. PCT/EP2009/053640 filed Mar. 27, 2009; which claims the benefit of U.S. Provisional Application No. 61/086,891 filed Aug. 7, 2008 and 61/040,796 filed Mar. 31, 2008, which are incorporated herein in their entirety.

The present invention relates to drug fusions and conjugates that have improved serum half lives. These fusions and conjugates comprise immunoglobulin (antibody) single variable domains and GLP and/or exendin molecules. The invention further relates to uses, formulations, compositions and devices comprising such drug fusions and conjugates.

### BACKGROUND OF THE INVENTION

Many drugs that possess activities that could be useful for therapeutic and/or diagnostic purposes have limited value because they are rapidly eliminated from the body when administered. For example, many polypeptides that have therapeutically useful activities are rapidly cleared from the circulation via the kidney. Accordingly, a large dose must be administered in order to achieve a desired therapeutic effect. A need exists for improved therapeutic and diagnostic agents that have improved pharmacokinetic properties.

One such class of drugs that have a short half life in the body or systemic circulation is the incretin hormones such as Glucagon-like peptide 1, or Peptide YY and also exendin, for example exendin-4.

Glucagon-like peptide (GLP)-1 is an incretin hormone with potent glucose-dependent insulinotropic and glucagonostatic actions, trophic effects on the pancreatic β cells, and inhibitory effects on gastrointestinal secretion and motility, which combine to lower plasma glucose and reduce glycemic excursions. Furthermore, via its ability to enhance satiety, GLP-1 reduces food intake, thereby limiting weight gain, and may even cause weight loss. Taken together, these actions give GLP-1 a unique profile, considered highly desirable for an antidiabetic agent, particularly since the glucose dependency of its antihyperglycemic effects should minimize any risk of severe hypoglycemia. However, its pharmacokinetic/pharmacodynamic profile is such that native GLP-1 is not therapeutically useful. Thus, while GLP-1 is most effective when administered continuously, single subcutaneous injections have short-lasting effects. GLP-1 is highly susceptible to enzymatic degradation in vivo, and cleavage by dipeptidyl peptidase IV (DPP-IV) is probably the most relevant, since this occurs rapidly and generates a noninsulinotropic metabolite. Strategies for harnessing GLP-1's therapeutic potential, based on an understanding of factors influencing its metabolic stability and pharmacokinetic/pharmacodynamic profile, have therefore been the focus of intense research.

Extensive work has been done to attempt to inhibit the peptidase or to modify GLP-1 in such a way that its degradation is slowed down while still maintaining biological activity. WO05/027978 discloses GLP-1 derivatives having a protracted profile of action. WO 02/46227 discloses heterologous fusion proteins comprising a polypeptide (for example, albumin) fused to GLP-1 or analogues (the disclosure of these analogues is incorporated herein by reference as examples of GLP-1 analogues that can be used in the present invention). WO05/003296, WO03/060071, WO03/059934 disclose amino fusion protein wherein GLP-1 has fused with albumin to attempt to increase the half-life of the hormone.

However, despite these efforts a long lasting active GLP-1 has not been produced.

As such, particularly in the fields of diabetes and obesity, there is a tremendous need for improved GLP-1 peptides or other agents such as exendin-4 that similarly have an insulinotropic effect amenable to treatment for diabetes and obesity in particular. There is thus a need to modify GLP-1, exendin-4 and other insulinotropic peptides to provide longer duration of action in vivo while maintaining their low toxicity and therapeutic advantages.

### SUMMARY OF THE INVENTION

The present invention provides a composition which is a fusion or conjugate and which comprises or consists of (a) an insulinotropic agent or molecule, or an incretin drug or molecule, which can for example be an exendin-4, or a GLP-1 e.g. the GLP-1 (7-37) A8G mutant, present as a fusion or conjugate with (b) the DOM 7h-14 (Vk) domain antibody (dAb) which binds specifically to serum albumin, (the amino acid sequence of DOM 7h-14 is shown in FIG. 1(h): SEQ ID NO 8).

An amino acid or chemical linker may also optionally be present joining the insulinotropic agent or incretin drug, e.g. exendin-4 and/or GLP-1, with the dAb e.g. with the DOM7h-14 dAb. The linker can be for example a helical linker e.g. the helical linker of sequence shown in FIG. 1 (k): SEQ ID NO 11, or it may be a gly-ser linker e.g. with an amino acid sequence shown in FIG. 1 (l): SEQ ID NO 12.

In certain embodiments, the fusions (or conjugates) of the invention can comprise further molecules e.g. further peptides or polypeptides.

The insulinotropic agent or incretin drug (e.g. exendin and/or GLP-1) can be present as a fusion (or conjugate) with either the N-terminal or C-terminal of the dAb.

In certain embodiments the invention provides a polypeptide comprising or consisting of a fusion molecule which is selected from the following:
(a) 2×GLP-1 (7-37) A8G DOM7h-14 dAb fusion (DAT0114, the amino acid sequence is shown in FIG. 1(a): SEQ ID NO 1),
(b) Exendin 4 (G4S linker)3 DOM7h-14 dAb fusion (DAT0115, the amino acid sequence is shown in FIG. 1(b): SEQ ID NO 2),
(c) Exendin 4-DOM7h-14 dAb fusion (DAT0116, the amino acid sequence is shown in FIG. 1(c): SEQ ID NO 3),
(d) Exendin 4, helical linker, DOM7h-14 dAb fusion (DAT0117, the amino acid sequence is shown in FIG. 1(d): SEQ ID NO 4),
(e) GLP-1 (7-37) A8G (G4S linker)3 DOM7h-14 dAb fusion (DAT0118, the amino acid sequence is shown in FIG. 1(e): SEQ ID NO 5),
(f) GLP-1 (7-37) A8G DOM7h-14 dAb fusion (DAT0119, the amino acid sequence is shown in FIG. 1(f): SEQ ID NO 6),
(g) GLP-1 (7-37) ABG, helical linker, DOM7h-14 dAb fusion (DAT0120, the amino acid sequence is shown in FIG. 1(g): SEQ ID NO 7).
The invention also provides conjugate molecules with the amino acid sequences of those described above i.e. those with the amino acid sequences shown by SEQ ID NOs-1-7.
Dom 7h-14 is a human immunoglobulin single variable domain or dAb (Vk) that binds to serum albumin and its amino acid sequence is shown in FIG. 1(h): SEQ ID NO 8. The CDR regions of Dom7h-14 dAb are underlined in the amino acid sequence shown in FIG. 1(h): SEQ ID NO 8.

As used herein, “fusion” refers to a fusion protein that comprises as a first moiety a DOM7h-14 dAb that binds serum albumin and as a second moiety an insulinotropic agent or an incretin drug. The dAb that binds serum albumin and the drug or agent are present as discrete parts (moieties) of a single continuous polypeptide chain. The first (dAb) and second (incretin drug or insulinotropic agent) moieties can be directly bonded to each other through a peptide bond or linked through a suitable amino acid, or peptide or polypeptide linker. Additional moieties e.g. peptides or polypeptides (e.g. third, fourth) and/or linker sequences, can be present as appropriate. The first moiety can be in an N-terminal location, C-terminal location or internal relative to the second moiety. In certain embodiments the fusion protein contains one or more than one (e.g. one to about 20) dAb moieties.

As used herein, “conjugate” refers to a composition comprising a dAb that binds serum albumin to which an insulinotropic agent or incretin drug is covalently or non-covalently bonded. The insulinotropic agent or incretin drug can be covalently bonded to the dAb directly or indirectly through a suitable linker moiety. The drug or agent can be bonded to the dAb at any suitable position, such as the amino-terminus, the carboxyl-terminus or through suitable amino acid side chains (e.g., the ε amino group of lysine, or thiol group of cysteine). Alternatively, the drug or agent can be noncovalently bonded to the dAb directly (e.g., electrostatic interaction, hydrophobic interaction) or indirectly (e.g., through noncovalent binding of complementary binding partners (e.g., biotin and avidin), wherein one partner is covalently bonded to drug or agent and the complementary binding partner is covalently bonded to the dAb).

The invention further provides (substantially) pure monomer of any of the conjugates or fusions of the invention e.g. of DAT0114, DAT 0115, DAT0116, DAT0117, DAT0118, DAT0119 and DAT120. In one embodiment, it is at least 98, 99, 99.5% pure or 100% pure monomer.

The invention also provides nucleic acids encoding the fusions described herein for example nucleic acids encoding DAT0114, DAT 0115, DAT0116, DAT0117, DAT0118, DAT0119 and DAT120 and e.g. wherein the nucleic acid sequences are shown in FIG. 2 (SEQ ID NOS 13-23). Also provided are host cells that comprise these nucleic acids.

The invention further provides a method for producing a fusion of the present invention which method comprises maintaining a host cell that comprises a recombinant nucleic acid and/or construct that encodes a fusion of the invention under conditions suitable for expression of said recombinant nucleic acid, whereby a fusion is produced.

The invention also provides compositions (e.g., pharmaceutical compositions) comprising a fusion or conjugate of the invention.

The invention also provides a method for treating an individual having a disease or disorder, such as those described herein e.g. a metabolic disease such as hyperglycemia, impaired glucose tolerance, beta cell deficiency, diabetes (for example type 1 or type 2 diabetes or gestational diabetes) or obesity or diseases characterised by overeating e.g. it can be used to suppress appetite e.g. in Prader-Willi syndrome, and which comprises administering to said individual a therapeutically effective amount of a fusion or conjugate of the invention.

Other metabolic disorders include, but are not limited to, insulin resistance, insulin deficiency, hyperinsulinemia, hyperglycemia, dyslipidemia, hyperlipidemia, hyperketonemia, hypertension, coronary artery disease, atherosclerosis, renal failure, neuropathy (e.g., autonomic neuropathy, parasympathetic neuropathy, and polyneuropathy), retinopathy, cataracts, metabolic disorders (e.g., insulin and/or glucose metabolic disorders), endocrine disorders, obesity, weight loss, liver disorders (e.g., liver disease, cirrhosis of the liver, and disorders associated with liver transplant), and conditions associated with these diseases or disorders.

In addition, conditions associated with diabetes that can be prevented or treated with the compounds of the present invention include, but are not limited to, hyperglycemia, obesity, diabetic retinopathy, mononeuropathy, polyneuropathy, atherosclerosis, ulcers, heart disease, stroke, anemia, gangrene (e.g., of the feet and hands), impotence, infection, cataract, poor kidney function, malfunctioning of the autonomic nervous system, impaired white blood cell function, Carpal tunnel syndrome, Dupuytren's contracture, and diabetic ketoacidosis.

The invention also provides methods for treating or preventing diseases associated with elevated blood glucose comprising administering at least one dose of the conjugates of fusions and/or pharmaceutical compositions of the present invention to patient or subject.

The invention further relates to methods of regulating insulin responsiveness in a patient, as well as methods of increasing glucose uptake by a cell, and methods of regulating insulin sensitivity of a cell, using the conjugates or fusions of the invention. Also provided are methods of stimulating insulin synthesis and release, enhancing adipose, muscle or liver tissue sensitivity towards insulin uptake, stimulating glucose uptake, slowing digestive process, or blocking the secretion of glucagon in a patient, comprising administering to said patient a fusion or conjugate of the invention e.g. comprising administering at least one dose of the drug conjugate or fusions and/or pharmaceutical composition of the present invention.

The fusions or conjugates and/or pharmaceutical compositions of the invention may be administered alone or in combination with other molecules or moieties e.g. polypeptides, therapeutic proteins and/or molecules (e.g., insulin and/or other proteins (including antibodies), peptides, or small molecules that regulate insulin sensitivity, weight, heart disease, hypertension, neuropathy, cell metabolism, and/or glucose, insulin, or other hormone levels, in a patient). In specific embodiments, the conjugates or fusions of the invention are administered in combination with insulin (or an insulin derivative, analog, fusion protein, or secretagogue).

The invention also provides for use of a conjugate or fusion of the invention for the manufacture of a medicament for treatment of a disease or disorder, such as any of those mentioned above e.g. a metabolic disorder such as hyperglycemia, diabetes (type 1 or 2 or gestational diabetes) or obesity.

The invention also relates to use of a fusion or conjugate as described herein for use in therapy, diagnosis or prophylaxis.

The fusions or conjugates of the invention e.g. the dAb component of the fusion can be further formatted to have a larger hydrodynamic size to further extend the half life, for example, by attachment of a PEG group, serum albumin, transferrin, transferrin receptor or at least the transferrin-binding portion thereof, an antibody Fc region, or by conjugation to an antibody domain. For example, the dAb that binds serum albumin can be formatted as a larger antigen-binding fragment of an antibody (e.g., formatted as a Fab, Fab′, F(ab)₂, F(ab′)₂, IgG, scFv).

In other embodiments of the invention described throughout this disclosure, instead of the use of a “dAb” in a fusion of the invention, it is contemplated that the skilled addressee can use a domain that comprises the CDRs of a dAb e.g. CDRs of Dom7h-14, that binds serum albumin (e.g., CDRs grafted onto a suitable protein scaffold or skeleton, eg an affibody, an SpA scaffold, an LDL receptor class A domain or an EGF domain). The disclosure as a whole is to be construed accordingly to provide disclosure of such domains in place of a dAb.

In certain embodiments, the invention provides a fusion or conjugate according to the invention that comprises an insulinotropic agent or increting drug and a dual-specific ligand or multi-specific ligand that comprises a first dAb according to the invention that binds serum albumin e.g. Dom7h-14, and a second dAb that has the same or a different binding specificity from the first dAb and optionally in the case of multi-specific ligands further dAbs. The second dAb (or further dAbs) may optionally bind a different target e.g. FgFr 1c, or CD5 target.

Thus, in one aspect, the invention provides the fusions or conjugates of the invention for delivery by parenteral administration e.g. by subcutaneous, intramuscular or intravenous injection, inhalation, nasal delivery, transmucossal delivery, oral delivery, delivery to the GI tract of a patient, rectal delivery or ocular delivery. In one aspect, the invention provides the use of the fusions or conjugates of the invention in the manufacture of a medicament for delivery by subcutaneous injection, inhalation, intravenous delivery, nasal delivery, transmucossal delivery, oral delivery, delivery to the GI tract of a patient, rectal delivery or ocular delivery.

In one aspect, the invention provides a method for delivery to a patient by subcutaneous injection, pulmonary delivery, intravenous delivery, nasal delivery, transmucossal delivery, oral delivery, delivery to the GI tract of a patient, rectal or ocular delivery, wherein the method comprises administering to the patient a pharmaceutically effective amount of a fusion or conjugate of the invention.

In one aspect, the invention provides an oral, injectable, inhalable, nebulisable or ocular formulation comprising a fusion or conjugate of the invention. The formulation can be a tablet, pill, capsule, liquid or syrup. In one aspect the compositions can be administered orally e.g. as a drink, for example marketed as a weight loss drink for obesity treatment. In one aspect, the invention provides a formulation for rectal delivery to a patient, the fomulation can be provided e.g. as a suppository.

A composition for parenteral administration of GLP-1 compounds may, for example, be prepared as described in WO 03/002136 (incorporated herein by reference).

A composition for nasal administration of certain peptides may, for example, be prepared as described in European Patent No. 272097 (to Novo Nordisk A/S) or in WO 93/18785 (all incorporated herein by reference).

The term “subject” or “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species.

The invention also provides a kit for use in administering compositions according to the invention (e.g., conjugates or fusions of the invention) to a subject (e.g., patient), comprising a composition (e.g., conjugate or fusion of the invention), a drug delivery device and, optionally, instructions for use. The composition (e.g., conjugate, or fusion) can be provided as a formulation, such as a freeze dried formulation. In certain embodiments, the drug delivery device is selected from the group consisting of a syringe, an inhaler, an intranasal or ocular administration device (e.g., a mister, eye or nose dropper), and a needleless injection device.

The compositions (e.g conjugates or fusions) of this invention can be lyophilized for storage and reconstituted in a suitable carrier prior to use. Any suitable lyophilization method (e.g., spray drying, cake drying) and/or reconstitution techniques can be employed. It will be appreciated by those skilled in the art that lyophilisation and reconstitution can lead to varying degrees of antibody activity loss and that use levels may have to be adjusted to compensate. In a particular embodiment, the invention provides a composition comprising a lyophilized (freeze dried) composition (e.g., drug conjugate, drug fusion) as described herein. Preferably, the lyophilized (freeze dried) composition (e.g., drug conjugate, drug fusion) loses no more than about 20%, or no more than about 25%, or no more than about 30%, or no more than about 35%, or no more than about 40%, or no more than about 45%, or no more than about 50% of its activity (e.g., binding activity for serum albumin) when rehydrated. Activity is the amount of composition (e.g., drug conjugate, drug fusion) required to produce the effect of the s composition before it was lyophilized. For example, the amount of conjugate or fusion needed to achieve and maintain a desired serum concentration for a desired period of time. The activity of the composition (e.g., drug conjugate, drug fusion) can be determined using any suitable method before lyophilization, and the activity can be determined using the same method after rehydration to determine amount of lost activity.

The invention also provides sustained release formulations comprising the fusions or conjugates of the invention, such sustained release formulations can comprise the fusion or conjugate of the invention in combination with, e.g. hyaluronic acid, microspheres or liposomes and other pharmaceutically or pharmacalogically acceptable carriers, excipients and/or diluents. Such sustained release formulations can in the form of for example suppositories.

In one aspect, the invention provides a pharmaceutical composition comprising a fusion or conjugate of the invention, and a pharmaceutically or physiologically acceptable carrier, excipient or diluent.

### BRIEF DESCRIPTION OF THE DRAWINGS

FIG. 1 is an illustration of the amino acid sequences of (a) DAT0114 (SEQ ID NO 1), (b) DAT0115 (SEQ ID NO 2), (c) DAT0116 (SEQ ID NO 3), (d) DAT0117 (SEQ ID NO 4), (e) DAT0118 (SEQ ID NO 5), (f) DAT0119 (SEQ ID NO 6) (g) DAT0120 (SEQ ID NO 7) (h) Dom7h-14 (SEQ ID NO 8) (dAb) (the CDRs are underlined), (i) GLP-1 7-37 A(8)G (SEQ ID NO 9), (j) exendin-4 (SEQ ID NO 10), (k) Helical linker (SEQ ID NO 11) (l) Gly-ser linker (SEQ ID NO 12).

FIG. 2 is an illustration of the nucleic acid sequences of (a) DAT0114 (mammalian construct) (SEQ ID NO 13), (b) DAT0115 (mammalian construct) (SEQ ID NO 14), (c) DAT0115 (optimized for E. coli construct) (SEQ ID NO 15), (d) DAT0116 (mammalian construct) (SEQ ID NO 16), (e) DAT0116 (optimized for E. coli construct) (SEQ ID NO 17), (f) DAT0117 (mammalian construct) (SEQ ID NO 18), (g) DAT0117 (optimized for E. coli construct) (SEQ ID NO 19), (h) DAT0118 (mammalian construct) (SEQ ID NO 20), (i) DAT0119 (mammalian construct) (SEQ ID NO 21), (j) DAT0120 (mammalian construct) (SEQ ID NO 22), (k) Dom7h-14 (SEQ ID NO 23).

FIG. 3 (a) shows dose dependent reduction in body weight in mouse model of obesity by administering DAT0115 (b) shows daily food consumption in mouse model of obesity by administering DAT0115.

FIG. 4 shows a DSC of DAT0115: Solid line—DAT0115 trace, dotted line—fit to a non-2-state model.

FIG. 5 shows a DSC of Lysozyme: Solid line—lysozyme trace, dotted line—fit to a non-2-state model (traces overlay so dotted trace cannot be seen).

FIG. 6 shows SEC MALLS of DAT0115.

FIG. 7 shows SEC MALLS of DAT0117.

FIG. 8 shows SEC MALLS of DAT0120.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

The term “insulinotropic agent” as used herein means a compound which is able to stimulate, or cause the stimulation of, the synthesis or expression of, or the activity of the hormone insulin. Known examples of insulinotropic agents include but are not limited to e.g. glucose, GIP, GLP, Exendin (e.g. exendin-4 and exendin-3), PYY and OXM.

The term “incretin” as used herein means a type of gastrointestinal hormone that causes an increase in the amount of insulin released when glucose levels are normal or particularly when they are elevated. By way of example they include GLP-1, GIP, OXM, PYY, VIP, and PP (pancreatic polypeptide).

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide or they can be within the peptide. A simple system is used to describe analogues of GLP-1: For example GLP-1 A8G (7-37 amino acids) designates a GLP-1 analogue wherein the naturally occurring alanine at position 8 has been substituted with a glycine residue. Formulae of peptide analogs and derivatives thereof are drawn using standard single letter abbreviation for amino acids used according to IUPAC-IUB nomenclature.

As used herein “fragment,” when used in reference to a polypeptide, is a polypeptide having an amino acid sequence that is the same as part but not all of the amino acid sequence of the entire naturally occurring polypeptide. Fragments may be “free-standing” or comprised within a larger polypeptide of which they form a part or region as a single continuous region in a single larger polypeptide. By way of example, a fragment of naturally occurring GLP-1 would include amino acids 7 to 36 of naturally occurring amino acids 1 to 36. Furthermore, fragments of a polypeptide may also be variants of the naturally occurring partial sequence. For instance, a fragment of GLP-1 comprising amino acids 7-30 of naturally occurring GLP-1 may also be a variant having amino acid substitutions within its partial sequence.

Examples of suitable insulinotropic agents of the invention include GLP-1, GLP-1 derivatives, GLP-1 analogues, or a derivative of a GLP-1 analogue. In addition they include Exendin-4, Exendin-4 analogues and Exendin-4 derivatives or fragments and Exendin-3, Exendin-3 derivatives and Exendin-3 analogues.

The term “GLP-1” as used herein means GLP-1 (7-37), GLP-1 (7-36), GLP-1 (7-35), GLP-1 (7-38), GLP-1 (7-39), GLP-1 (7-40), GLP-1 (7-41), a GLP-1 analogue, a GLP-1 peptide, a GLP-1 derivative or mutant or fragment or a derivative of a GLP-1 analogue. Such peptides, mutants, analogues and derivatives are insulinotropic agents.

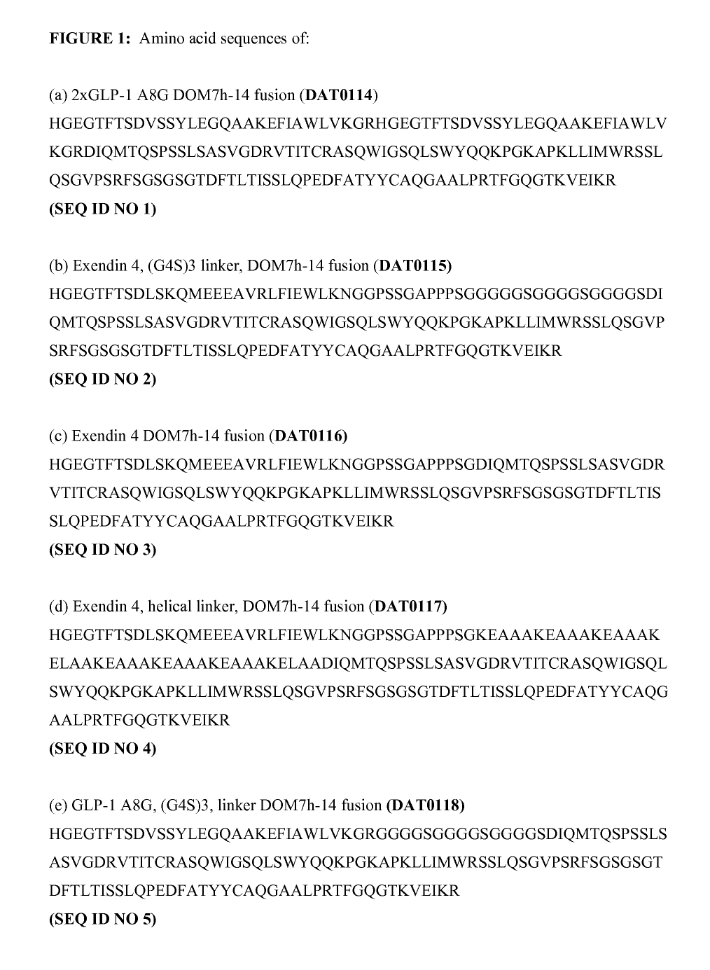

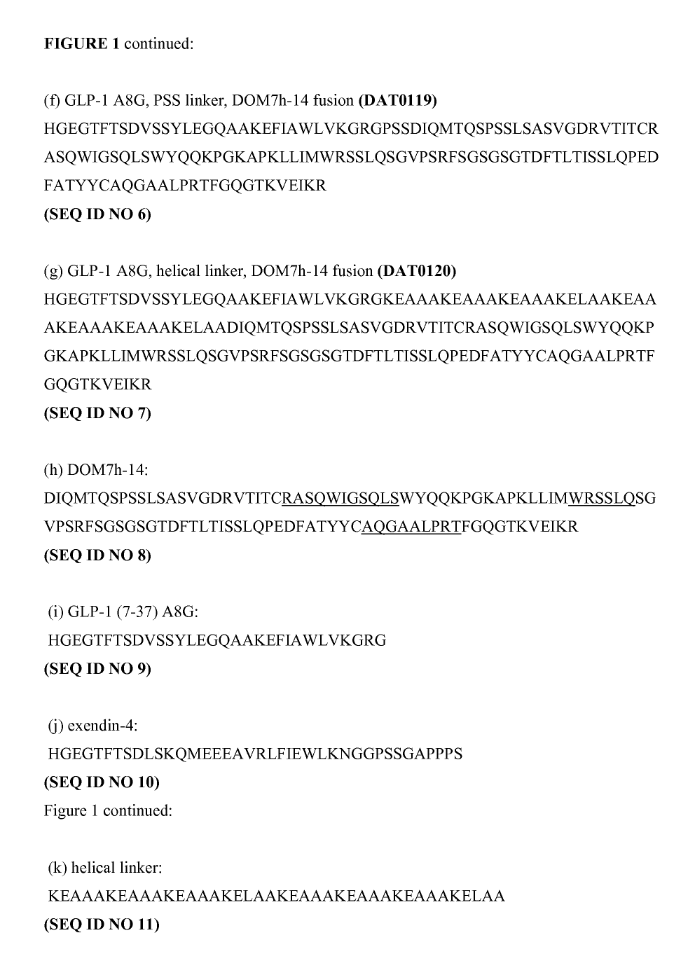

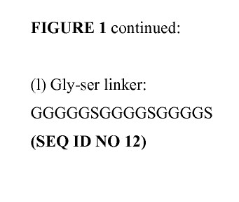

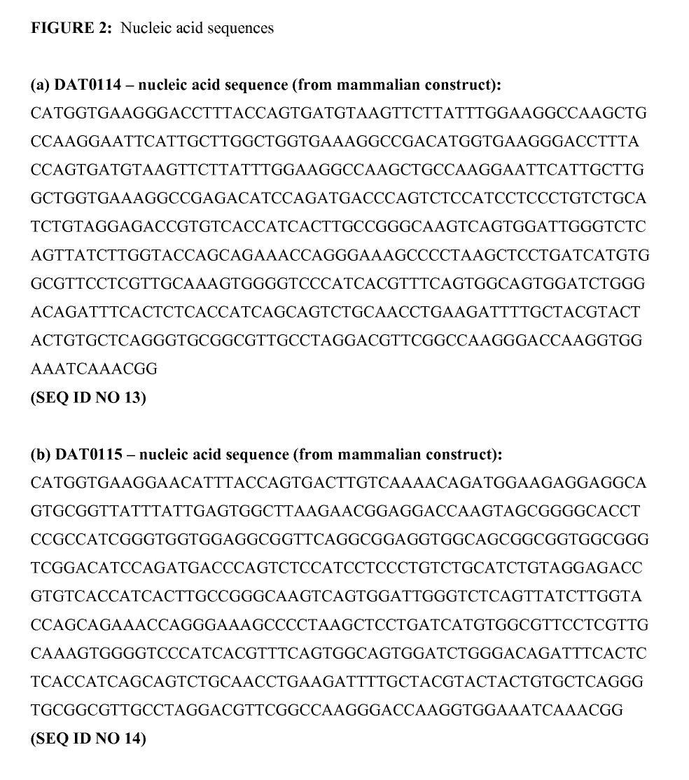

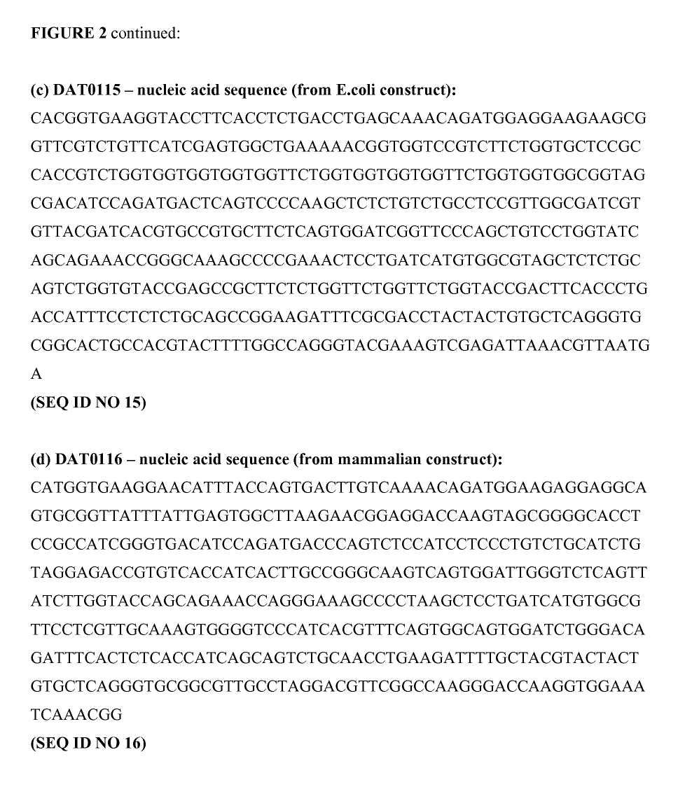

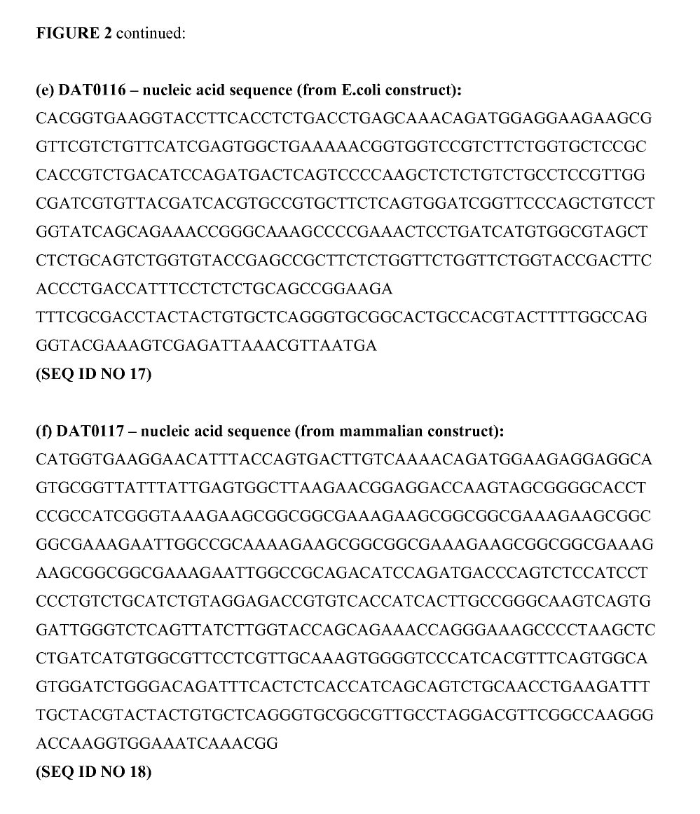

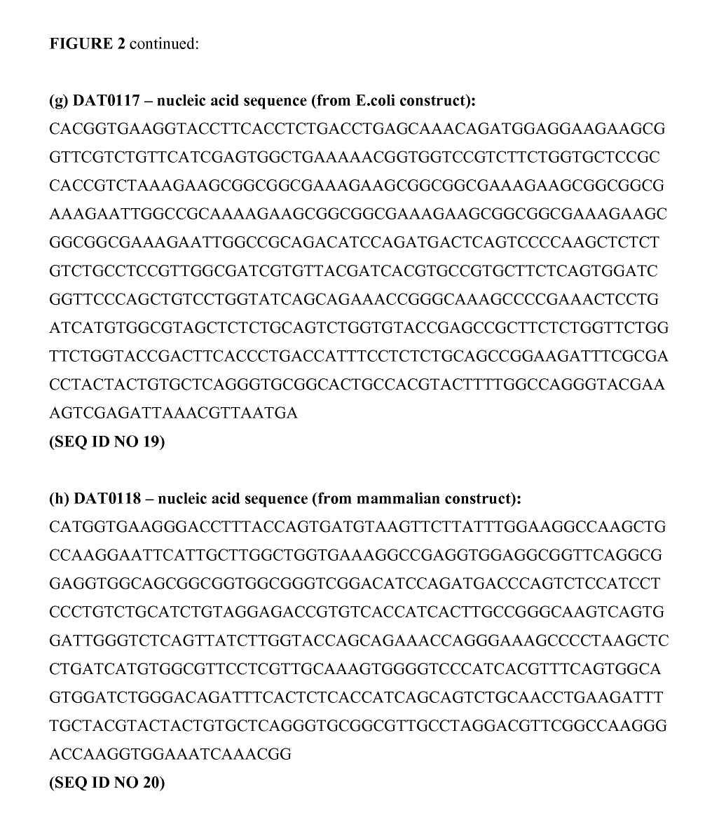

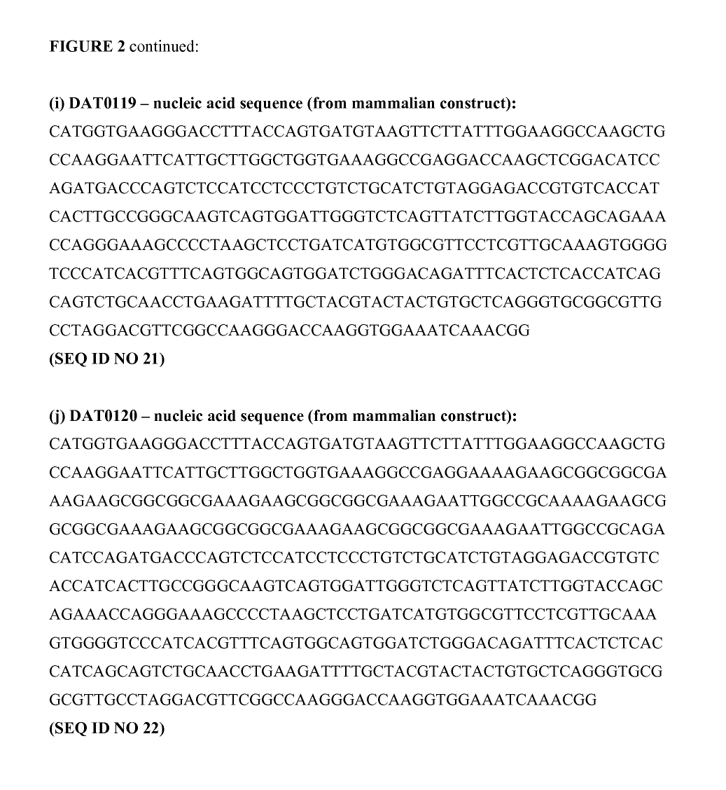

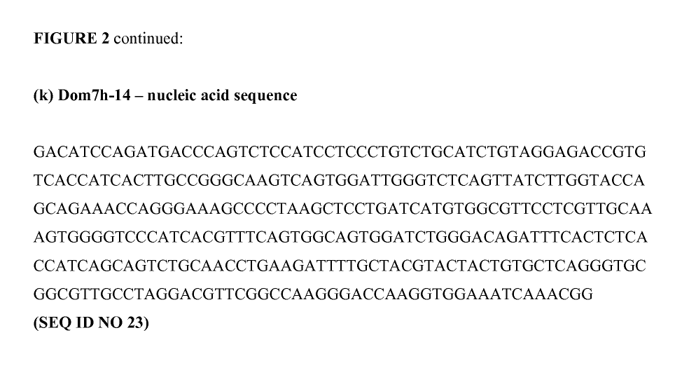

For example the GLP-1 can be GLP-1 (7-37) A8G mutant with the amino acid sequence shown in FIG. 1 (i): SEQ ID NO 9.

Further GLP-1 analogues are described in International Patent Application No. 90/11296 (The General Hospital Corporation) which relates to peptide fragments which comprise GLP-1 (7-36) and functional derivatives thereof and have an insulinotropic activity which exceeds the insulinotropic activity of GLP-1 (1-36) or GLP-1 (1-37) and to their use as insulinotropic agents (incorporated herein by reference, particularly by way of examples of drugs for use in the present invention).

International Patent Application No. WO 91/11457 (Buckley et al.) discloses analogues of the active GLP-1 peptides 7-34, 7-35, 7-36, and 7-37 which can also be useful as GLP-1 drugs according to the present invention (incorporated herein by reference, particularly by way of examples of drugs or agents for use in the present invention).

The term “exendin-4 peptide” as used herein means exendin-4 (1-39), an exendin-4 analogue, a fragment of exendin-4 peptide, an exendin-4 derivative or a derivative of an exendin-4 analogue. Such peptides, fragments, analogues and derivatives are insulinotropic agents. The amino acid sequence of exendin-4 (1-39) is shown in FIG. 1 (j): SEQ ID NO 10.

Further Exendin-analogs that are useful for the present invention are described in PCT patent publications WO 99/25728 (Beeley et al.), WO 99/25727 Beeley et al.), WO 98/05351 (Young et al.), WO 99/40788 (Young et al.), WO 99/07404 (Beeley et al), and WO 99/43708 (Knudsen et al) (all incorporated herein by reference, particularly by way of examples of drugs for use in the present invention).

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

As used herein, “display system” refers to a system in which a collection of polypeptides or peptides are accessible for selection based upon a desired characteristic, such as a physical, chemical or functional characteristic. The display system can be a suitable repertoire of polypeptides or peptides (e.g., in a solution, immobilized on a suitable support). The display system can also be a system that employs a cellular expression system (e.g., expression of a library of nucleic acids in, e.g., transformed, infected, transfected or transduced cells and display of the encoded polypeptides on the surface of the cells) or an acellular expression system (e.g., emulsion compartmentalization and display). Exemplary display systems link the coding function of a nucleic acid and physical, chemical and/or functional characteristics of a polypeptide or peptide encoded by the nucleic acid. When such a display system is employed, polypeptides or peptides that have a desired physical, chemical and/or functional characteristic can be selected and a nucleic acid encoding the selected polypeptide or peptide can be readily isolated or recovered. A number of display systems that link the coding function of a nucleic acid and physical, chemical and/or functional characteristics of a polypeptide or peptide are known in the art, for example, bacteriophage display (phage display, for example phagemid display), ribosome display, emulsion compartmentalization and display, yeast display, puromycin display, bacterial display, display on plasmid, covalent display and the like. (See, e.g., EP 0436597 (Dyax), U.S. Pat. No. 6,172,197 (McCafferty et al.), U.S. Pat. No. 6,489,103 (Griffiths et al.))

As used herein, “functional” describes a polypeptide or peptide that has biological activity, such as specific binding activity. For example, the term “functional polypeptide” includes an antibody or antigen-binding fragment thereof that binds a target antigen through its antigen-binding site.

As used herein, “target ligand” refers to a ligand which is specifically or selectively bound by a polypeptide or peptide. For example, when a polypeptide is an antibody or antigen-binding fragment thereof, the target ligand can be any desired antigen or epitope. Binding to the target antigen is dependent upon the polypeptide or peptide being functional.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (V_{H}, V_{HH}, V_{L}) that specifically binds an antigen or epitope independently of other V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one embodiment, each individual organism or cell contains only one or a limited number of library members. In one embodiment, the nucleic acids are incorporated into expression vectors, in order to allow expression of the polypeptides encoded by the nucleic acids. In an aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

As used herein, the term “dose” refers to the quantity of fusion or conjugate administered to a subject all at one time (unit dose), or in two or more administrations over a defined time interval. For example, dose can refer to the quantity of fusion or conjugate administered to a subject over the course of one day (24 hours) (daily dose), two days, one week, two weeks, three weeks or one or more months (e.g., by a single administration, or by two or more administrations). The interval between doses can be any desired amount of time.

The phrase, “half-life,” refers to the time taken for the serum or plasma concentration of the fusion or conjugate to reduce by 50%, in vivo, for example due to degradation and/or clearance or sequestration by natural mechanisms. The fusions or conjugates of the invention are stabilized in vivo and their half-life increased by binding to serum albumin molecules e.g. human serum albumin (HSA) which resist degradation and/or clearance or sequestration. These serum albumin molecules are naturally occurring proteins which themselves have a long half-life in vivo. The half-life of a molecule is increased if its functional activity persists, in vivo, for a longer period than a similar molecule which is not specific for the half-life increasing molecule. For example, a fusion or conjugate of the invention comprising a dAb specific for human serum albumin (HSA) and an incretin drug or insulinotropic agent such as GLP-1 or exendin is compared with the same ligand wherein the specificity to HSA is not present, that is does not bind HSA but binds another molecule. For example, it may bind a third target on the cell. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible.

As used herein, “hydrodynamic size” refers to the apparent size of a molecule (e.g., a protein molecule, ligand) based on the diffusion of the molecule through an aqueous solution. The diffusion, or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein.

Calculations of “homology” or “identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In an embodiment, the length of a reference sequence aligned for comparison purposes is at least 30%, or at least 40%, or at least 50%, or at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences. Amino acid and nucleotide sequence alignments and homology, similarity or identity, as defined herein may be prepared and determined using the algorithm BLAST 2 Sequences, using default parameters (Tatusova, T. A. et al., FEMS Microbiol Lett, 174:187-188 (1999).

### Nucleic Acids, Host Cells:

The invention relates to isolated and/or recombinant nucleic acids encoding the fusions of the invention that are described herein e.g. those encoded by SEQ ID NOS 13-23.

Nucleic acids referred to herein as “isolated” are nucleic acids which have been separated away from other material (e.g., other nucleic acids such as genomic DNA, cDNA and/or RNA) in its original environment (e.g., in cells or in a mixture of nucleic acids such as a library). An isolated nucleic acid can be isolated as part of a vector (e.g., a plasmid).

Nucleic acids referred to herein as “recombinant” are nucleic acids which have been produced by recombinant DNA methodology, including methods which rely upon artificial recombination, such as cloning into a vector or chromosome using, for example, restriction enzymes, homologous recombination, viruses and the like, and nucleic acids prepared using the polymerase chain reaction (PCR).

The invention also relates to a recombinant host cell e.g. mammalian or microbial, which comprises a (one or more) recombinant nucleic acid or expression construct comprising a nucleic acid encoding a fusion of the invention as described herein. There is also provided a method of preparing a fusion of the invention as described herein, comprising maintaining a recombinant host cell e.g. mammalian or microbial, of the invention under conditions appropriate for expression of the fusion polypeptide. The method can further comprise the step of isolating or recovering the fusion, if desired.

For example, a nucleic acid molecule (i.e., one or more nucleic acid molecules) encoding a fusion polypeptide of the invention, or an expression construct (i.e., one or more constructs) comprising such nucleic acid molecule(s), can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g., in the presence of an inducer, in a suitable animal, in suitable culture media supplemented with appropriate salts, growth factors, antibiotics, nutritional supplements, etc.), whereby the encoded peptide or polypeptide is produced. If desired, the encoded peptide or polypeptide can be isolated or recovered (e.g., from the animal, the host cell, medium, milk). This process encompasses expression in a host cell of a transgenic animal (see, e.g., WO 92/03918, GenPharm International).

The fusion polypeptides of the invention described herein can also be produced in a suitable in vitro expression system, e.g. by chemical synthesis or by any other suitable method.

As described and exemplified herein, the fusions or conjugates of the invention generally bind serum albumin with high affinity.

For example, the fusions or conjugates of the invention can bind human serum albumin with an affinity (KD; KD=K_{off}(kd)/Kₒₙ (ka) [as determined by surface plasmon resonance) of about 5 micromolar to about 100 pM, e.g. about 1 micromolar to about 100 pM e.g. 400-800 nm e.g. about 600 nm.

The fusion or conjugates of the invention can be expressed in E. coli or in Pichia species (e.g., P. pastoris). In one embodiment, the fusion is secreted in a quantity of at least about 0.5 mg/L when expressed in E. coli or in Pichia species (e.g., P. pastoris); or in mammalian cell culture (e.g. CHO, or HEK 293 cells). Although, the fusions or conjugates described herein can be secretable when expressed in E. coli or in Pichia species or mammalian cells they can be produced using any suitable method, such as synthetic chemical methods or biological production methods that do not employ E. coli or Pichia species.

In certain embodiments, the fusions and conjugates of the invention are efficacious in animal models of such as those described in WO 2006/059106 (e.g. at pages 104-105 of published WO 2006/059106) or those described in the examples herein, when an effective amount is administered. Generally an effective amount is about 0.0001 mg/kg to about 10 mg/kg (e.g., about 0.001 mg/kg to about 10 mg/kg, e.g. about 0.001 mg/kg to about 1 mg/kg, e.g. about 0.01 mg/kg to about 1 mg/kg, e.g. about 0.01 mg/kg to about 0.1 mg/kg). The models of disease are recognized by those skilled in the art as being predictive of therapeutic efficacy in humans.

Generally, the present fusions and conjugates of the invention will be utilised in purified form together with pharmacologically or physiologically appropriate carriers. Typically, these carriers can include aqueous or alcoholic/aqueous solutions, emulsions or suspensions, any including saline and/or buffered media. Parenteral vehicles can include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The route of administration of pharmaceutical compositions according to the invention may be any of those commonly known to those of ordinary skill in the art. For therapy, the drug fusions or conjugates of the invention can be administered to any patient in accordance with standard techniques.

The administration can be by any appropriate mode, including parenterally, intravenously, intramuscularly, intraperitoneally, orally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. Administration can be local or systemic as indicated.

In one embodiment, the invention provides a pulmonary formulation for delivery to the lung which comprises (a) the conjugates and fusions of the invention, and (b) a pharmaceutically acceptable buffer, and wherein the composition comprises liquid droplets and about 40% or more e.g. 50% or more, of the liquid droplets present in the composition have a size in the range which is less than about 6 microns e.g. from about 1 micron to about 6 microns e.g. less than about 5 microns e.g. about 1 to about 5 microns These compositions are e.g. especially suitable for administration to a subject by direct local pulmonary delivery. These compositions can, for example, be administered directly to the lung, e.g. by inhalation, for example by using a nebuliser device. These compositions for pulmonary delivery can comprise a physiologically acceptable buffer, which has a pH range of between about 4 to about 8, e.g. about 7 to about 7.5, and a viscosity which is about equal to the viscosity of a solution of about 2% to about 10% PEG 1000 in 50 mM phosphate buffer containing 1.2% (w/v) sucrose.

The fusions or conjugates of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

For prophylactic applications, e.g. when administering to individuals with pre-diabetes or with insulin resistance, compositions containing the present fusions or conjugates may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a fusion or conjugate of the invention is administered to treat, suppress or prevent disease, it can be administered up to four times per day, twice weekly, once weekly, once every two weeks, once a month, or once every two months, at a dose of, for example about 0.0001 mg/kg to about 10 mg/kg (e.g., about 0.001 mg/kg to about 10 mg/kg e.g. about 0.001 mg/kg to about 1 mg/kg e.g. about 0.01 mg/kg to about 1 mg/kg, e.g. about 0.01 mg/kg to about 0.1 mg/kg).

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the precise nature of the disease or disorder targeted, but can be measured by an ordinarily skilled clinician or technician.

Similarly, prophylaxis performed using a composition as described herein is “effective” if the onset or severity of one or more symptoms is delayed, reduced or abolished relative to such symptoms in a similar individual (human or animal model) not treated with the composition.

The fusions and conjugates of the present invention may be used as separately administered compositions or they may be administered in conjunction with other therapeutic or active agents e.g. other polypeptides or peptides or small molecules. These further agents can include various drugs, such as for example metformin, insulin, glitazones (e.g. rosaglitazone), immunosuppresives, immunostimulants.

The fusions and conjugates of the invention can be administered and/or formulated together with one or more additional therapeutic or active agents. When a fusion or conjugate of the invention is administered with an additional therapeutic agent, the fusion or conjugate can be administered before, simultaneously, with, or subsequent to administration of the additional agent. Generally, the fusion or conjugate of the invention and the additional agent are administered in a manner that provides an overlap of therapeutic effect.

### Half-Life:

Increased half-life of the insulinotropic agent or incretin drug e.g. the GLP-1 or exendin ligand is useful in in vivo applications. The invention solves this problem by providing increased half-life of the insulinotropic agent or incretin drug e.g. GLP and exendin, in vivo and consequently longer persistence times in the body of the functional activity of these molecules.

As described herein, compositions of the invention (i.e. comprising the fusions or conjugates described herein) can have dramatically prolonged in vivo serum or plasma half-life and/or increased AUC and/or increased mean residence time (MRT), as compared to insulinotropic agent or incretin drug alone. In addition, the activity of the insulinotropic agent or incretin drug is generally not substantially altered in the composition of the invention (e.g., the conjugate, or the fusion). However, some change in the activity of compositions of the invention compared to insulinotropic agent or incretin drug alone is acceptable and is generally compensated for by the improved pharmacokinetic properties of the conjugates or fusions of the invention. For example, drug conjugates or fusions of the invention may bind the drug target with lower affinity than drug alone, but have about equivalent or superior efficacy in comparison to drug alone due to the improved pharmacokinetic properties (e.g., prolonged in vivo serum half-life, larger AUC) of the drug composition. In addition, due to the increased half life the conjugates or fusions of the invention they can be administed less frequently than the insulinotropic agent or incretin drug alone e.g. they can be given to patients once a month or once a week, and they also attain a more constant level of insulinotropic agent or incretin drug in the blood than administration of insulinotropic agent or incretin drug alone, so achieving the desired therapeutic or prophylactic effect.

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Half lives (t½ alpha and VA beta) and AUC and MRT can be determined from a curve of plasma or serum concentration of ligand against time. The WinNonlin analysis package (available from Pharsight Corp., Mountain View, Calif.94040, USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The t alpha half life is the half life of the first phase and the t beta half life is the half life of the second phase. In addition a non-compartmental fitting model that is well known in the art can also be used to determine half life.

In one embodiment, the present invention provides a fusion or conjugate according to the invention that has an elimination half-life e.g. in human subjects, in the range of about 12 hours or more, e.g. about 12 hours to about 21 days, e.g. about 24 hours to about 21 days, e.g. about 2-8 days e.g. about 3-4 days.

The fusions or conjugates of the invention can also be further formatted to have a larger hydrodynamic size, for example, by attachment of a PEG group, serum albumin, transferrin, transferrin receptor or at least the transferrin-binding portion thereof, an antibody Fc region, or by conjugation to an antibody domain.

Hydrodynamic size may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

Compositions of the invention, i.e. those comprising the fusions and conjugates described herein, provide several further advantages. The Domain antibody component is very stable, is small relative to antibodies and other antigen-binding fragments of antibodies, can be produced in high yields by expression in E. coli or yeast (e.g., Pichia pastoris), and antigen-binding fragments of antibodies that bind serum albumin can be easily selected from libraries of human origin or from any desired species. Accordingly, compositions of the invention that comprise the dAb that binds serum albumin can be produced more easily than therapeutics that are generally produced in mammalian cells (e.g., human, humanized or chimeric antibodies) and dAbs that are not immunogenic can be used (e.g., a human dAb can be used for treating or diagnosing disease in humans).

The immunogenicity of the insulinotropic agent or incretin drug can be reduced when the insulinotropic agent or incretin is part of a drug composition that contains a dAb binds serum albumin. Accordingly, the invention provides a fusion or conjugate compositions which can be less immunogenic (than e.g. the insulinotropic agent or incretin alone) or which can be substantially non-immunogenic in the context of a drug composition that contains a dAb that binds serum albumin. Thus, such compositions can be administered to a subject repeatedly over time with minimal loss of efficacy due to the elaboration of anti-drug antibodies by the subject's immune system.

Additionally, the conjugate or fusion compositions described herein can have an enhanced safety profile and fewer side effects than the insulinotropic agent or incretin alone. For example, as a result of the serum albumin-binding activity of the dAb, the fusions and conjugates of the invention have enhanced residence time in the vascular circulation. Additionally, the fusions and conjugates of the invention are substantially unable to cross the blood brain barrier and to accumulate in the central nervous system following systemic administration (e.g., intravascular administration). Accordingly, the fusions or conjugates of the invention can be administered with greater safety and reduced side effects in comparison to the insulinotropic agent or incretin drug alone. Similarly, the fusions or conjugates can have reduced toxicity toward particular organs (e.g., kidney or liver) than drug alone.

### EXAMPLES

### Example 1

### Expression of Genetic Fusions of GLP-1 (A8G) or Exendin-4 and DOM7h-14 AlbudAb

Either exendin-4 or GLP-1 (7-37), with alanine at position 8 replaced by glycine ([Gly⁸]GLP-1), was cloned as a fusion with DOM7h-14 (a domain antibody (dAb) which binds serum albumin (albudab) with an amino acid sequence shown below) into the pTT-5 vector (obtainable from CNRC, Canada). In each case the GLP-1 or exendin-4 was at the 5′ end of the construct and the dAb at the 3′ end. In total, 7 constructs (DAT0114, DAT 0115, DAT0116, DAT 0117, DAT 0118, DAT 0119, DAT 0120) were made with the amino acid sequences shown in FIG. 1 (A-G). There was either no linker, a gly-ser linker (G4S), or a helical linker (Arai, R., H. Ueda, et al. (2001). “Design of the linkers which effectively separate domains of a bifunctional fusion protein.” Protein Eng 14(8): 529-32.456) or a linker composed of a second GLP-1 moiety between the GLP-1 or exendin 4 and the dAb. The linkers were included as spacers to separate the GLP-1 or exendin 4 spatially from the dAb to prevent steric hinderence of the binding between the GLP-1 or exendin-4 and the GLP-1 receptor. The sequences of the constructs are shown in FIG. 1 (A-G).
Endotoxin free DNA was prepared in E. coli using alkaline lysis (using the endotoxin free plasmid Giga kit, obtainable from Qiagen Calif.) and used to transfect HEK293E cells (obtainable from CNRC, Canada). Transfection was into 250 ml/flask of HEK293E cells at 1.75×10⁶ cells/ml using 333 ul of 293fectin (Invotrogen) and 250 ug of DNA per flask and expression was at 30° C. for 5 days. The supernatant was harvested by centrifugation and purification was by affinity purification on protein L. Protein was batch bound to the resin, packed on a column and washed with 10 column volumes of PBS. Protein was eluted with 50 ml of 0.1M glycine pH2 and neatralised with Tris pH8. Protein of the expected size was identified on an SDS-PAGE gel and sizes are shown in the table 1 below

TABLE 1
Molecular weights of DAT0114, DAT 0115, DAT0116,
DAT 0117, DAT 0118, DAT 0119, DAT 0120 constructs
as determined on SDS-PAGE gel

| Fusion protein | Expected MW |
|---|---|
| DAT0114 | 18256 |
| DAT0115 | 16896 |
| DAT0116 | 15950 |
| DAT0117 | 19798 |
| DAT0118 | 15936 |
| DAT0119 | 15318 |
| DAT0120 | 18895 |

### Example 2

### Showing that GLP-1 and Exendin-4 AlbudAb Fusions Bind Serum Albumin

GLP-1 and Exendin-4 AlbudAb fusions were analysed by surface plasmon resonance (Biacore AB obtainable from GE Healthcare) to obtain information on affinity. The analysis was performed using a CMS Biacore chip (carboxymethylated dextran matrix) that was coated with serum albumin. About 1000 resonance units (RUs) of each serum albumin to be tested (human, rat and mouse serum albumin) was immobilised in acetate buffer pH 5.5. Flow cell 1 of the Biocore AB was an uncoated, blocked negative control, flow cell 2 was coated with Human serum albumin (HSA) (815 RUs) flow cell 3 was coated with Rat serum albumin (RSA)(826RUs) and flow cell 4 was coated with Mouse serum albumin (MSA) (938 RUs). Each fusion molecule tested was expressed in mammalian tissue culture as described in the example above.
A range of concentrations of the fusion molecule were prepared (in the range 16 nM to 2 μM) by dilution into BIACORE HBS-EP buffer (0.01M HEPES, pH7.4, 0.15M NaCl, 3 mM EDTA, 0.005% surfactant P20) and flowed across the BIACORE chip. Affinity (KD) was calculated from the BIACORE traces by fitting on-rate and off-rate curves to traces generated by concentrations of dAb in the region of the KD. Affinities (KD) are summarised in the following table 2:

TABLE 2
Binding of GLP-1 and exendin-4 AlbudAb
to human, rat and mouse serum albumins

| | GLP-1 (7-37) A8G, helical | 2xGLP-1 (7-37) A8G |
|---|---|---|
| | linker, DOM7h-14 fusion | DOM7h-14 fusion |
| HSA | 110 nM | 150 nM |
| RSA | 800 nM | 700 nM |
| MSA | 110 nM | 130 nM |

The results above demonstrate that the fusion molecules retain the ability to bind to all types of serum albumin and this indicates that they are likely to have an extended half life in vivo.

### Example 3

### GLP-1 and Exendin-4 AlbudAb Fusions are Active in a GLP-1 Receptor Binding Assay (GLP-1R BA)

Fusions were buffer exchanged into 100 mM NaV1, 20 mM citrate pH 6.2. Meanwhile, CHO 6CRE GLP1R cells (CHO K1 cells (obtainable from the American Type Tissue Collection, ATCC) stably transfected with 6 cAMP response element driving a luciferase reporter gene and also with the human GLP-1 receptor) were seeded at 2×10⁵ cells/mL in suspension media. Suspension culture was maintained for 24 hours. Cells were then diluted into 15 mM HEPES buffer (obtainable from Sigma), containing 2 mM L glutamine (2.5×10⁵ cells/ml) and dispensed into 384-well plates containing 10 ul/well of the compound to be assayed. After the addition of assay control, plates were returned to the incubator for 3 h at 37° C. and 5% CO2. After the incubation, steady glo luciferase substrate (obtainable from Promega) was added to the wells as described in the kit and the plates sealed with self-adhesive plate seals (Weber Marking Systems Inc. Cat. No. 607780). Plates were placed in the reader (Viewlux, Perkin Elmer) and pre-incubated for 5 minutes prior to reading the fluorescence and plotting of results. Compound was assayed at a range of concentrations in the presence and absence of 10 uM albumin, allowing a dose response curve to be fitted with and without the albumin. EC50s were calculated and are summarised in the following table 3:

TABLE 3
Activity of GLP-1 and exendin-4 AlbudAb fusions
in a GLP-1 receptor binding assay (GLP-1RBA)

| | GLP-1R BA | GLP-1R BA |
|---|---|---|
| | EC₅₀ (pM) | (10 uM albumin) |
| | n = 3 | EC₅₀ (pM) n = 2 |
| Exendin 4 (G4S)3 | 8.9 | 35 |
| DOM7h-14 fusion | | |
| Exendin 4 | 12 | 72 |
| DOM7h-14 fusion | | |
| Exendin 4, helical | 4.3 | 15 |
| linker, DOM7h-14 | | |
| fusion | | |
| GLP-1 A8G, | 17 | 130 |
| helical linker, | | |
| DOM7h-14 fusion | | |
| GLP-1 7-36 | 21 | 19 |
| Exendin-4 | 1.0 | 0.82 |

The results above demonstrate that all of the fusion molecules tested retain potency for binding to the GLP-1 receptor. The results also demonstrate that this potency is retained in the presence of serum albumin. Hence, these fusion molecules are likely to retain the ability to bind the GLP-1 receptor in vivo.

### Example 4

### Expression of DAT0115, DAT0116, DAT0117 and DAT0120 in HEK 293 Mammalian Tissue Culture Followed by Purification by Protein L Affinity Capture and Ion Exchange Chromatography

The aim of this experiment was to produce protein for in vivo and in vitro characterisation. Protein was expressed in mammalian tissue culture in HEK 293E cells from the pTT-5 vector as described in the previously. Briefly, endotoxin free DNA was prepared and purified and used to transfect HEK293E cells. Protein expression was for 5 days at 30° C. in a shaking incubator and cultures were spun down and supernatant (containing the protein of interest) harvested. Protein was purified from the supernatant by affinity capture on protein L agarose streamline affinity resin (resin GE Healthcare, protein L coupled in house). Resin was then washed with 10 column volumes of PBS and then protein was eluted with 5 column volumes of 0.1M glycine pH2.0. Neutralisation was with 1 column volume of 1M Tris glycine pH8.0. In this case (contrasting with the previous example), further purification was then undertaken. Protein (in tris-glycine) was buffer exchanged to 20 mM acetate pH 5.0 prior to loading using the Akta onto 1 (or 2 in parallel) 6 ml resource S columns (GE healthcare) pre-equilibrated in 20 mM acetate pH 5.0. After washing with the same buffer, protein was eluted via a 0-0.75M or 0-1M NaCl gradient in 20 mM acetate pH5.0. Fractions of the correct size were then identified by SDS-PAGE electrophoresis and by mass spectrometry and were then combined to make the final protein sample. Protein was then buffer exchanged into 20 mM citrate, pH6.2, 100 mM NaCl and concentrated to between 0.5 and 5 mg/ml. Protein was filtered through a 0.2 uM filter to ensure sterility. Protein was then used in examples described below.

### Example 5

### Comparison of the Stability of DAT0115, DAT0116, DAT0117 and DAT0120 to 1, 3 and 6 Freeze Thaw Cycles

The aim of this study was to compare the stability of DAT0115, DAT0116, DAT0117 and DAT0120 to 1, 3, and 6 freeze thaw cycles. Each protein was expressed in mammalian tissue culture in HEK 293E cells from the pTT-5 vector and purified on protein L affinity resin followed by ion exchange chromatography as described above. Protein was buffer exchanged into 20 mM citrate, 100 mM NaCl and diluted to 0.5 mg/ml using the same buffer. 0.5 ml aliquots of each protein (in eppendorf tubes) were then subjected to 0, 1, 3 or 6 freeze thaw cycles, with each cycle comprising 3 minutes on dry ice followed by 2 minutes in a 37° C. water bath. (It was observed during the experiment that 2 minutes at 37° C. was sufficient for the protein solution to completely thaw.) After completion of the requisite number of freeze-thaw cycles, protein samples were stored at 2-8° C. until further analysis. Proteins were then subjected to analysis by SDS PAGE electrophoresis, GLP-1R binding assay, size exclusion chromatography on a Superdex 75 column and mass spectrometry. It was observed that SDS-PAGE profile, potency by GLP-1R BA and mass spec profile of all four protein was not significantly changed from baseline by 1, 3 or 6 freeze-thaw cycles. Maximum peak height in the SEC analysis was affected with 78%, 86%, 104% and 57% of maximum height maintained after 6 freeze thaw cycles for DAT0115, DAT-116, DAT0117 and DAT0120 respectively. It was concluded that DAT0120 was less stable to freeze thaw cycles than the other three proteins.

TABLE 4
Results of comparison of the stability of DAT0115, DAT0116,
DAT0117 and DAT0120 to 1, 3 and 6 freeze thaw cycles

| | Number of | | |
|---|---|---|---|
| | freeze thaw | Peak height | % max peak |
| Sample | cycles | (mAU) | height |
| DAT0115 | 0 | 49 | 100% |
| DAT0115 | 1 | 43 | 89% |
| DAT0115 | 3 | 40 | 82% |
| DAT0115 | 6 | 38 | 78% |
| DAT0116 | 0 | 29 | 100% |
| DAT0116 | 1 | 28 | 95% |
| DAT0116 | 3 | 26 | 91% |
| DAT0116 | 6 | 25 | 86% |
| DAT0117 | 0 | 34 | 100% |
| DAT0117 | 1 | 34 | 99% |
| DAT0117 | 3 | 35 | 103% |
| DAT0117 | 6 | 35 | 104% |
| DAT0120 0 | 0 | 35 | 100% |
| DAT0120 1 | 1 | 24 | 70% |
| DAT0120 3 | 3 | 21 | 59% |
| DAT0120 6 | 6 | 20 | 57% |

### Example 6

### Demonstration of the Duration of Action of DAT0115 in the Db/Db Mouse Model of Type II Diabetes

The aim of this study was to determine the duration of action of DAT0115 on oral glucose tolerance in db/db mice. Animals were sorted by decreasing glucose levels three days prior to the start of the experiment and then blocked. One animal within each block was then assigned to each of the 26 study groups. This ensured similar mean starting glucose level in each of the study groups.
DAT0115 (produced in HEK293 cells and purified as described above) was administered subcutaneously at 1 mg/Kg, 0.3 mg/Kg or 0.1 mg/Kg either 5 h, 24 h, 48 h, 72 h, 96 h or 120 h hours prior to the oral glucose load. (Not all doses were administered at every timepoint, see table below for details.) DAT0115 significantly decreased the glucose AUC over the 2 hour time period of the oral-glucose tolerance test (OGTT) compared to vehicle treated db/db mice at timpoints out to and including 24 h for the 0.1 mg/Kg and 0.3 mg/Kg doses and out to and including the 72 h timepoint for the 1 mg/Kg dose. Exendin-4, administered as a positive control at 42 μg/Kg, also significantly reduced the glucose AUC following OGTT when administered 5 h prior to the oral glucose bolus. The table 5 below shows the percentage reduction in AUC for each of the DAT0115 study groups compared to vehicle. An asterisk indicates P<0.05 for DAT0115 comparison to vehicle using the false discovery rate correction.

TABLE 5
showing the percentage reduction in AUC for each of
the DAT0115 study groups compared to vehicle. (An asterisk
indicates P < 0.05 for DAT0115 comparison to vehicle
using the false discovery rate correction)

| OGTT Time (hrs | 0.1 mg/kg | 0.3 mg/kg | 1 mg/kg |
|---|---|---|---|
| relative to dosing) | DAT0115 | DAT0115 | DAT0115 |
| +5 | 60%* | Not done | 76%* |
| +24 | 36%* | 59%* | 50%* |
| +48 | 28% | 26% | 37%* |
| +72 | 16% | 26% | 41%* |
| +96 | −12% | Not done | 12% |

### Example 7

### Demonstration of Efficacy of DAT0115 in the Diet Induced Obese (DIO) Mouse Model of Obesity

The aim of this study was to use an established mouse feeding model (diet induced obese mice) to determine whether food consumption and, as a result, body weight is affected by treatment with DAT0115. This may be predictive for humans. Male C57B1/6 mice (purchased from Taconic) were fattened on 60% kcal high fat irradiated diet for 12 wks and then transferred to the in-house facility. Upon arrival, the mice were individually housed on alpha-dri bedding in a temperature and humidity controlled room (70-72° F., Humidity=48-50%, 5 AM/5 PM light cycle). The diet was changed to 45% high fat diet and the animals acclimated for 18 days. Prior to administration of test compound, mice were injected subcutaneously with saline once daily for three days and food consumption monitored. Mice were blocked and grouped such that body weight and food consumption were not different between or within groups. On the day of the study, groups of 8 mice were dosed subcutaneously as follows using a 5 ml/kg injection volume: Three groups were dosed with DAT0115 (low, medium and high dose), one group with a negative control molecule (DOM7h-14 AlbudAb, but with no exendin-4 conjugate) and one with exendin-4 positive control.

TABLE 6
Protocol for Establishing Efficacy of DAT0115 in the
diet induced obese (DIO) mouse model of obesity

| Group | Compound administered | Dose level | |
|---|---|---|---|
| 1 | Negative control: DOM7h-14 in 100 mM NaCl, | 1 | mg/Kg |
| | 20 mM citrate/sodium citrate pH 6.2 | | |
| 2 | Exendin-4 | 0.01 | mg/Kg |
| 3 | DAT0115 in 100 mM NaCl, 20 mM citrate/ | 0.01 | mg/Kg |
| | sodium citrate pH 6.2 | | |
| 4 | DAT0115 in 100 mM NaCl, 20 mM citrate/ | 0.1 | mg/Kg |
| | sodium citrate pH 6.2 | | |
| 5 | DAT0115 in 100 mM NaCl, 20 mM citrate/ | 1 | mg/Kg |
| | sodium citrate pH 6.2 | | |

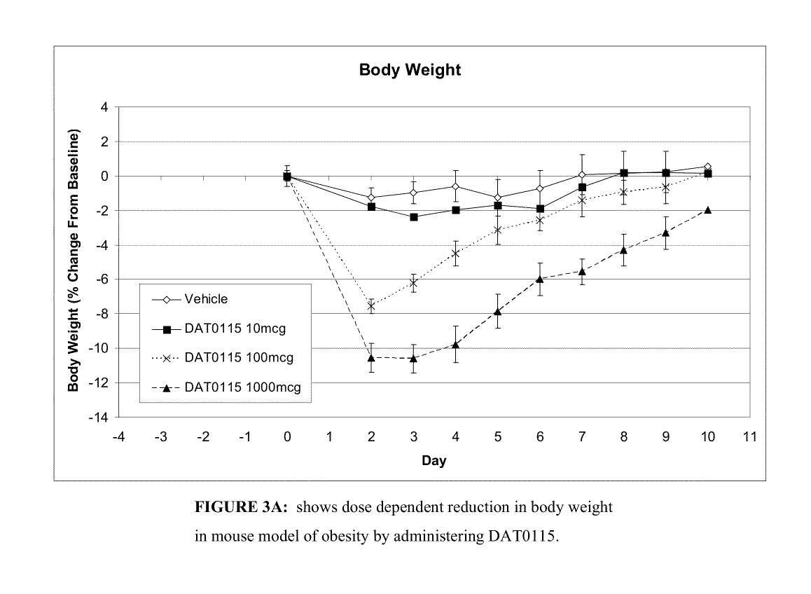

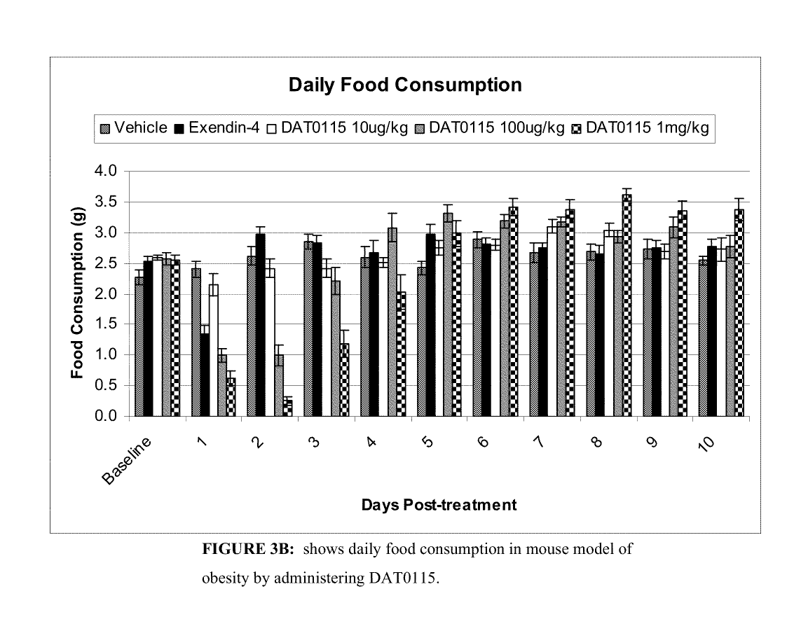

Daily food consumption and body weight were measured daily for 10 days. DAT0115 showed dose dependent reduction in body weight and food consumption compared to the DOM7h-14 control (see FIGS. 3a and 3b). It was therefore concluded that the data from this mouse study supports the hypothesis that DAT0115 would be a good clinical candidate.

### Example 8

### Determination of the Plasma Half Life of DAT0115, DAT0116 and DAT0117 in a Mouse Model of Type II Diabetes

The aim of this study was to determine a plasma elimination profile for DAT0115, DAT0116 and DAT0117 in a mouse model of type II diabetes (db/db mice) and to calculate PK parameters from the results. DAT0115, DAT0116 and DAT0117 protein was prepared as described earlier: Briefly, protein was expressed in mammalian tissue culture using HEK293E cells and purified using batch absorption to protein L-agarose affinity resin followed by elution with glycine at pH 2.0 and neutralisation woth Tris PH 8.0. This was followed by ion exchange chromotography on a Resource S column using a 0-1M salt gradient in 20 mM acetate pH5.0. Fractions containing the desired protein were then combined and buffer exchanged into 100 mM NaCl, 20 mM citrate pH6.2. Protein was filter sterilised, buffer exchanged and endotoxin removed ant tested prior to use in vivo. Groups of non-fasted male db/db mice (LEPr db homozygous mice deficient for the leptin receptor with mutations in the leptin receptor gene (lepr)) were dosed either subcutaneously or intravenously with 1 mg/Kg DAT0115, DAT0116 or DAT0117. At predose, 0.25, 0.5, 1, 4, 7, 12, 24, 36, 48 and 60 hours after dosing for the iv doses and predose, 0.5, 1, 4, 7, 12, 24, 36, 48 and 60 hours after dosing for the sc doses blood samples were collected by terminal bleed and plasma prepared. Plasma samples were frozen and later defrosted for analysis of DAT0115, DAT0116 or DAT0117 levels as appropriate by solid phase extraction and LC/MS/MS to detect the presence of a fragment of the protein (from the exendin-4 section of the protein). Calculated plasma levels were then used to fit pharmacokinetic parameters using WinNonLin software. Half life after subcutaneous and intravenous administration and bioavailability is outlined in the table below. It was concluded from the results (see table 7 below) that all three compounds show desirable pharmacokinetic parameters in a mouse model of type II diabetes. Therefore, these molecules show the potential for good PK parameters in diabetic humans, with this study favoring the choice of DAT0115 or DAT0116 over DAT0117.

TABLE 7
Plasma half life of DAT0115, DAT0116 and DAT0117
in a mouse model of type II diabetes

| | Half-life after | Half-life after | |
|---|---|---|---|
| | intravenous | subcutaneous | |
| Compound | administration | administration | Bioavailability |
| DAT0115 | 13.8 | 18.6 | 65% |
| DAT0116 | 14.3 | 20.1 | 61% |
| DAT0117 | 11.4 | 11.2 | 25% |

### Example 9

### Determination of the Plasma Half Life of DAT0115, DAT0116, DAT0117 in Rat

The aim of this study was to determine a plasma elimination profile for DAT0115, DAT0116 and DAT0117 in rat and to calculate PK parameters from the results. DAT0115, DAT0116 and DAT0117 protein was prepared as described earlier: Briefly, protein expressed in mammalian tissue culture using HEK293E cells and purified using batch absorption to protein L-agarose affinity resin followed by elution with glycine at pH 2.0 and neutralisation woth Tris pH 8.0. This was followed by ion exchange chromotography on a Resource S column using a 0-1M salt gradient in 20 mM acetate pH5.0. Fractions containing the desired protein were then combined and buffer exchanged into 100 mM NaCl, 20 mM citrate pH6.2. Protein was filter sterilised, buffer exchanged and Qced prior to use in vivo.

In order to determine plasma half life, groups of 3 rats were given a single i.v or s.c. injection at 0.3 mg/Kg (iv) or 1.0 mg/Kg (sc) of DAT0115, DAT0116 or DAT0117. Plamsa samples were obtained by serial bleeds from a tail vein over a 72 h period and analyzed by LC/MS/MS to detect the presence of a fragment of the fusion (from the exendin-4 section of the fusion). Calculated plasma levels were then used to fit pharmacokinetic parameters using WinNonLin software. Half life after subcutaneous and intravenous administration and bioavailability is outlined in the table 8 below. It was concluded from the results that all three compounds show desirable pharmacokinetic parameters in rat. Therefore, all these molecules show the potential for good PK parameters in humans, with this study favoring the choice of DAT0115 over DAT0116 or DAT0117.

TABLE 8
Half life after subcutaneous and intravenous
administration and bioavailability

| | Half-life after | half life after | |
|---|---|---|---|
| | intravenous | subcutaneous | |
| Compound | administration | administration | Bioavailability |
| DAT0115 | 4.9 h | 11.1 h | 81% |
| DAT0116 | 4.1 h | 8.7 h | 32% |
| DAT0117 | 4.9 h | 10.2 h | 15% |

### Example 10

### Determination of the Plasma Half Life of DAT0115 in Cynomolgus Monkey

The aim of this study was to determine the pharmacokinetic parameters for DAT0115 in a non human primate (cynomolgus monkey) to enable allometric scaling of parameters and give the best possible indication of whether DAT0115 was likely to have a good PK profile in humans. DAT0115 Exendin-4 AlbudAb fusion was expressed in HEK293E cells in mammalian tissue culture and purified as described earlier. Briefly, protein was purified using batch absorption to protein L-agarose affinity resin followed by elution with glycine at pH 2.0 and neutralisation woth Tris PH 8.0. This was followed by ion exchange chromotography on a Resource S column using a 0-1M salt gradient in 20 mM acetate pH5.0. Fractions containing the desired protein were then combined and buffer exchanged into 100 mM NaCl, 20 mM citrate pH6.2.

Protein was extensively QCed (including SDS-PAGE, mass spec, activity assay:GLP-1R-BA, pH check, osmolarity check), filter sterilised and endotoxin removed. Protein with confimed low endotoxin (<0.05 EU/mg protein) was then used for the in vivo study. Six female cynomolgus monkeys (Macaca fascicularis; Charles River Laboratories BRF, Houston, Tex., Primate Products, Miami, Fla. and/or Covance Research Products, Inc., Alice, Tex.) were used in this study. The monkeys were approximately 2 to 9 years old (with a body weight range of approximately 2 to 5 kilograms) at initiation of dosing. The monkeys were housed individually in stainless-steel cages in an environmentally controlled room(s) (64 F to 84 F; 30 to 70% relative humidity) with a 12-hour light/dark cycle. The female monkeys were offered approximately 6 biscuits twice daily of Monkey Diet #5038 (PMI Nutrition International, Richmond, Ind.) and a daily allotment of fresh fruit. Each animal was administered the test compound (DAT0115) either subcutaneously or intravenously according to dose group (3 sc and 3 iv). Dose was at 0.1 mg/Kg. On the days of dosing, the first feeding occurred within approximately 1 hour post dose for each monkey (extended up to 2.5 hours post dose if study-related procedures required animals to be out of their local housing for an extended period of time). The second feeding was no sooner then two hours following the first feeding. For the purpose of environmental enrichment, additional fruit, legume and/or vegetable (e.g., grapes, baby carrots, peanuts) was provided to each monkey at or around the time of viability check or as a method of reward after acclimation or study-related procedures. Filtered tap water (supplied by Aqua Pennsylvania, Inc. and periodically analyzed) was available ad libitum.
Plasma samples (approx 2 ml) were collected from the femoral vessel at predose (0 hour) and nominally at 5 minutes (iv group only), 0.5, 4, 8, 24, 48, 96, 144, 192, 288, 336, 504 and 672 hours after dosing. (PK samples from one of the animals in the iv dose group were only collected to 24 h so this animal has been excluded from the PK fitting). Analysis of samples was by mass spectrometry, and fitting of the data was using WinNonLin fitting software. PK parameters were as follows for iv administration (n=2): T_{1/2} 67 h, MRT 46 h, Vz 327 ml/Kg and Cl 3.3 ml/hr/Kg; and for sc administration (n=3): T_{1/2} 68 h, MRT 98 h, Vz 306 ml/Kg and Cl 3.1 ml/hr/Kg. Bioavailability was calculated as 99%.

It was concluded from this study (and from biacore binding data to cyno and human serum albumin) that the 68 h sc half life of DAT0115 in cyno (as described above) gives confidence that the half life of the same molecule in humans is likely to be sufficiently long to correlate with a requirement for weekly (or less frequent) dosing.

### Example 11

### Determination of PD of DAT0115 in Cynomolgus Monkey

A PK study was conducted in cynomolgus monkey as described above. The principal aim of this study was to determine the pharmacokinetic parameters for DAT0115 in cynomolgus monkey (as described in the previous example), but a secondary aim was to obtain an indication of the efficacy of the DAT0115 compound in the monkeys (without sufficient power in the study for statistical significance). To achieve this secondary aim, biscuit consumption by the monkeys was monitored during the course of the study. It was noted in the days following dosing that there was a trend towards reduction in food consumption in all of the monkeys. It was concluded that this was probably due to the well documented effect of the exendin-4 part of the molecule as an appetite suppressant. Hence, DAT0115 is shown to be active in vivo.

TABLE 9
Measurement of Biscuits consumed daily by cynomolgus
monkey (with DAT0115 dosing on day 1)

| Dose | Day −2 | Day −1 | Day 1 | Day 2 | Day 3 | Day 4 | Day 5 | Day 6 | Day 7 |
|---|---|---|---|---|---|---|---|---|---|
| 0.1 mg/Kg | 12 | 12 | 6 | 3 | 9 | 12 | 12 | 12 | 12 |
| (iv) | | | | | | | | | |
| 0.1 mg/Kg | 12 | 12 | 0 | 1 | 0 | 4 | 6 | 10 | 11 |
| (iv) | | | | | | | | | |
| 0.1 mg/Kg | 12 | 12 | 0 | 0 | 0 | 0 | 1 | 6 | 6 |
| (iv) | | | | | | | | | |
| 0.1 mg/Kg | 12 | 12 | 5 | 0 | 4 | 11 | 7 | 11 | 12 |
| (sc) | | | | | | | | | |
| 0.1 mg/Kg | 12 | 12 | 12 | 0 | 2 | 12 | 12 | 12 | 12 |
| (sc) | | | | | | | | | |
| 0.1 mg/Kg | 12 | 12 | 11 | 12 | 11 | 12 | 8 | 12 | 12 |
| (sc) | | | | | | | | | |

### Example 12

### DAT0115 Exendin-4 AlbudAb Fusion Binds Rat, Cynomolgus Monkey and Human Serum Albumin Using Surface Plasmon Resonance

DAT0115 was expressed and purified and then analysed by surface plasmon resonance (Biacore, GE Healthcare) to obtain information on affinity. The analysis was performed using a streptavidin chip (SA) coated with biotinylated serum albumin. 200-1000 resonance units (RUs) of each serum albumin was immobilised on the chip. Flow cell 1 was uncoated, flow cell 2 was coated with HSA, flow cell 3 was coated with RSA and flow cell 4 was coated with CSA. A range of concentrations of fusion was prepared (in the range 15.6 nM to 2 μM by dilution into BIACORE HBS-EP buffer (0.01M HEPES, pH7.4, 0.15M NaCl, 3 mM EDTA, 0.005% surfactant P20) and flowed across the BIACORE chip.

Affinity (KD) was calculated from the BIACORE traces by fitting on-rate and off-rate curves to traces generated by concentrations of dAb in the region of the KD. Affinities (KD) are summarised in the following table:

TABLE 10
Affinity (KD) of DAT0115

| Serum albumin types | DAT0115 | |
|---|---|---|
| HSA | 600 | nM |
| RSA | 2 | uM |
| CSA | 2 | uM |

### Example 13

### Characterisation of DAT0115 Thermal Denaturation by Differential Scanning Calorimetry (DSC)

The aim of this experiment was to monitor the thermal denaturation of DAT0115 by DSC (Differential Scanning calorimetry) using a capillary cell microcalorimeter VP-DSC (Microcal) equipped with an autosampler. Protein was dialysed overnight into 20 mM citrate pH6.2, 100 mM NaCl, filtered and then prepared at concentration of 1 mg/ml as determined by absorbance at 280 nm. Filtered dialysis buffer was used as a reference for all samples. DSC was performed at a heating rate of 180° C./hour. Before each sample a solution of 1% decon, and then buffer were injected to clean the cells and to provide instrumental baseline. Obtained traces were analysed using Origin 7 Microcal software. The DSC trace obtained from the reference buffer was subtracted from the sample trace. Precise molar concentration of the sample was used for calculations (performed automatically by Origin). Baseline setting for both upper and lower baselines linear regions before/after transition were selected and connected using cubic connect function. The resulting graph is fitted to non-2-state model, generating apparent Tm, and ΔH/ΔHv values.

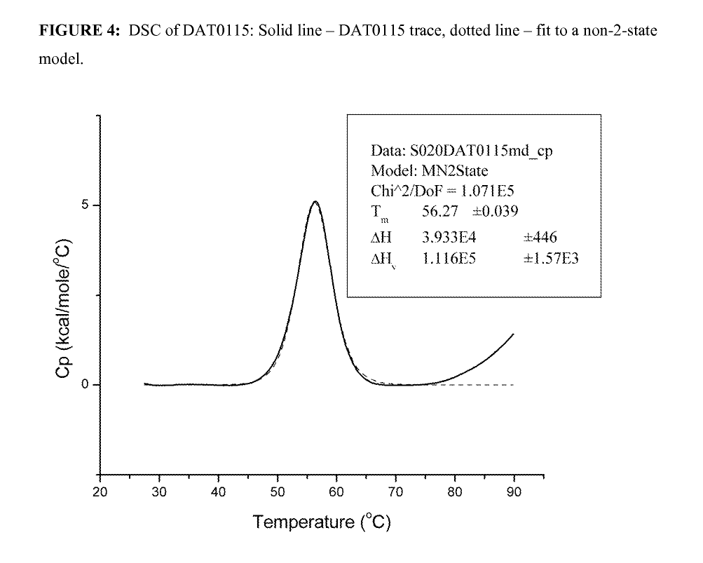

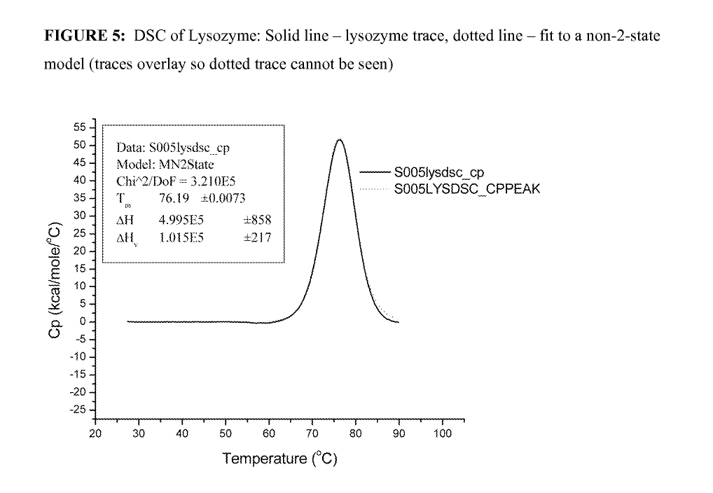

The trace from DAT0115 was fitted to a non-two state transition model, with appTm of 56.3° C. Goodness of fit was satisfactory (see FIG. 4). The control trace, lysozyme, run using the same equipment produced good quality data as expected with perfect fit. (AppTm obtained for lysozyme was 76.2° C. which is in agreement with that reported in the literature (see FIG. 5).) Therefore, it was concluded that this experiment had provided reliable data indicating that DAT0115 is a molecule with a melting temperature of 56.3° C. which is acceptable for a clinical candidate.

### Example 14

### Characterisation of DAT0115, DAT0117 and DAT0120 in Solution State by SEC MALLS

The aim of this experiment was to determine the in solution state of DAT0115, DAT0117 and DAT0120 by SEC MALLS. Samples were purified and dialysed into appropriate buffer (PBS) and filtered after dialysis, concentration was determined and adjusted to 1 mg/ml. BSA and HSA were purchased from Sigma and used without further purification.

### Details of Instrumentation:

Shimadzu LC-20AD Prominence HPLC system with an autosampler (SIL-20A) and SPD-20A Prominence UV/Vis detector was connected to Wyatt Mini Dawn Treos (MALLS, multi-angle laser light scattering detector) and Wyatt Optilab rEX DRI (differential refractive index) detector. The detectors were connected in the following order—LS—UV—RI. Both RI and LS instruments operated at a wavelength of 488 nm. TSK2000 (Tosoh corporation) or BioSep2000 (Phenomenex) columns were used (both are silica-based HPLC columns with similar separation range, 1-300 kDa) with mobile phase of 50 or 200 mM phosphate buffer (with or without salt), pH7.4 or 1×PBS. The flow rate used is 0.5 or 1 ml/min, the time of the run was adjusted to reflect different flow rates (45 or 23 min) and is not expected to have significant impact onto separation of the molecules. Proteins were prepared in PBS to a concentration of 1 mg/ml and injection volume was 100 ul. The light-scattering detector was calibrated with toluene according to manufacturer's instructions. The UV detector output and RI detector output were connected to the light scattering instrument so that the signals from all three detectors could be simultaneously collected with the Wyatt ASTRA software. Several injections of BSA in a mobile phase of PBS (0.5 or 1 ml/min) are run over a Tosoh TSK2000 column with UV, LS and RI signals collected by the Wyatt software. The traces are then analysed using ASTRA software, and the signals are normalised aligned and corrected for band broadening following manufacturer's instructions. Calibration constants are then averaged and input into the template which is used for future sample runs.

### Absolute Molar Mass Calculations:

100 ul of 1 mg/ml sample was injected onto appropriate pre-equilibrated column. After SEC column the sample passes through 3 on-line detectors—UV, MALLS (multi-angle laser light scattering) and DRI (differential refractive index) allowing absolute molar mass determination. The dilution that takes place on the column is about 10 fold, so the concentration at which in-solution state is determined is 100 ug/ml, or about 8 uM dAb.
The basis of the calculations in ASTRA as well as of the Zimm plot technique, which is often implemented in a batch sample mode is the equation from Zimm[J. Chem. Phys. 16, 1093-1099 (1948)]:

$$\frac{{R}_{θ}}{{K}^{*} c} = MP ( θ ) - 2 {A}_{2} {cM}^{2} {P}^{2} ( θ ) \qquad ( Eq . 1 )$$

where
c is the mass concentration of the solute molecules in the solvent (g/mL)
M is the weight average molar mass (g/mol)
A₂ is the second virial coefficient (mol mL/g²)
K*=4p²n₀²(dn/dc)²l₀⁻⁴N_{A}⁻¹ is an optical constant where n₀ is the refractive index of the solvent at the incident radiation (vacuum) wavelength, l₀ is the incident radiation (vacuum) wavelength, expressed in nanometers, N_{A} is Avogadro's number, equal to 6.022×10²³ mol⁻¹, and dn/dc is the differential refractive index increment of the solvent-solute solution with respect to a change in solute concentration, expressed in mL/g (this factor must be measured independently using a dRI detector).
P(q) is the theoretically-derived form factor, approximately equal to 1−2μ²<r²>/3!+ . . . , where μ=(4π/λ)sin(θ/2), and <r²> is the mean square radius. P(q) is a function of the molecules' z-average size, shape, and structure.
R_{q} is the excess Rayleigh ratio (cm⁻¹)
   This equation assumes vertically polarized incident light and is valid to order c².
   To perform calculations with the Zimm fit method, which is a fit to R_{q}/K^{*}c vs. sin²(q/2), we need to expand the reciprocal of Eq. 1 first order in c:
   To perform calculations with the Zimm fit method, which is a fit to
   Rq/K*c vs. sin 2(q/2), we need to expand the reciprocal of Eq. 1 to first order in c:

$$\frac{{K}^{*} c}{{R}_{θ}} = \frac{1}{MP ( θ )} + 2 {A}_{2} c \qquad Eq . 2$$

The appropriate results in this case are

$$\begin{matrix}M = {( \frac{{K}^{*} c}{{R}_{0}} - 2 {A}_{2} c )}^{- 1} \\ and & Eq . 3 \\ 〈 {r}^{2} 〉 = \frac{3 {m}_{0} {λ}^{2} M}{16 {π}^{2 }} & Eq . 4 \\ where {m}_{0} ≡ \frac{d [ {K}^{*} c / {R}_{θ} ]}{d {[ {sin}^{2} ( θ / 2 ) ]}_{θ -> 0}} & Eq . 5\end{matrix}$$

The calculations were performed automatically by ASTRA software, resulting in a plot with molar mass determined for eath of the slices [Astra manual].
Molar mass obtained from the plot for each of the peaks observed on chromatogram is compared with expected molecular mass of a single unit of the protein. This allows us to draw conclusions about in-solution state of the protein.

### Experimental Data

### DAT0115

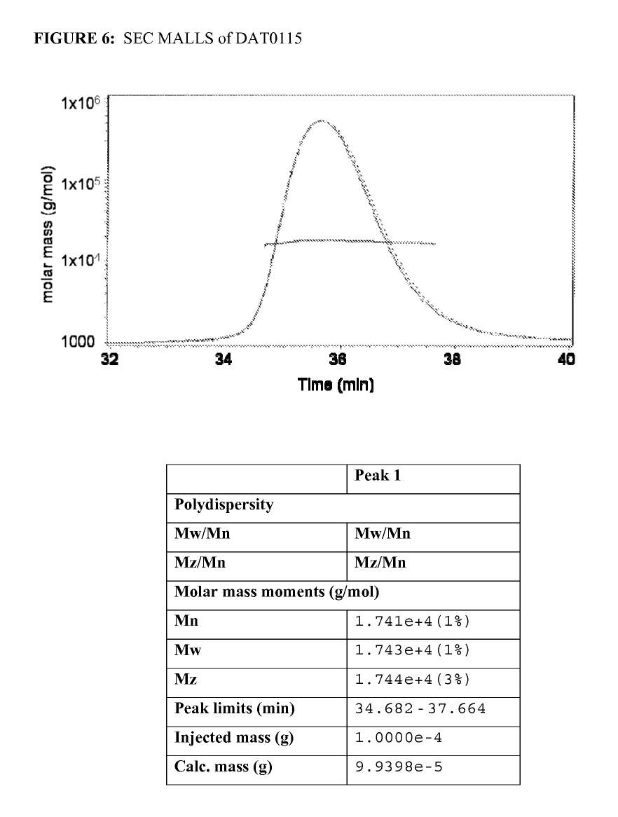

100 μl of 1 mg/ml DAT0115 was injected onto Superdex 200 column, equilibrated into 20 mM citrate, 0.1M NaCl, pH6.2. Flow speed was set at 0.5 ml/min. The protein eluted in a single peak, with Mw determined across the whole width of the peak as 17.4 kDa (expected Mw for a monomer is 16.9 kDa). Elution efficiency is 100%. See FIG. 6. (HSA control behaved as expected validating the experimental results for DAT0115. It elutes in two peaks with Mw 64 kDa (monomer) and 110 kDa (dimer). MW of the HSA dimer may not be very precise due to very small amount of protein within this peak.)

### DAT0117

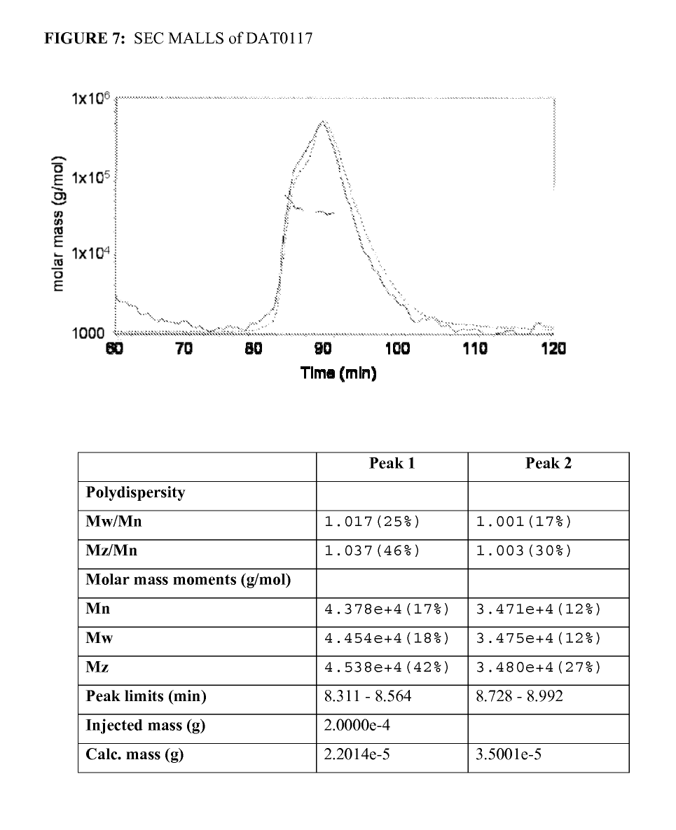

100 μl of 1 mg/ml DAT0117 was injected onto TSK2000 column, equilibrated into 50 mM phosphate buffer, pH7.4. Flow speed was set at 1 ml/min. About 50% of the injected amount of DAT0117 eluted off the column in two overlapping peaks with Mw around 35-45 kDa (dimer and above), which indicates strong self-association at the conditions tested here. (BSA control behaved as expected validating the DAT0117 experimental results, giving two peaks with molar mass of 61 kDa and 146 kDa (monomer and dimer). See FIG. 7 for SEC Mal results.

### DAT0120

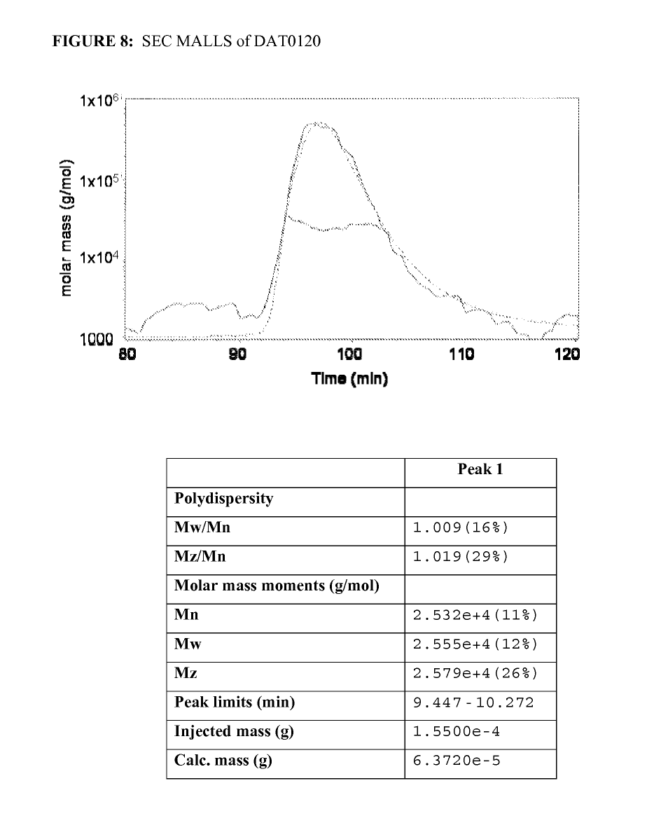

100 μl of 1 mg/ml DAT0117 was injected onto TSK2000 column, equilibrated into 50 mM phosphate buffer, pH7.4. Flow speed was set at 1 ml/min. About 50% of the injected amount of DAT0120 eluted off the GF column in slightly asymmetric peak with Mw determined at around 25 kDa. This indicates self-association of DAT0120 at the conditions tested here, the protein appears to be in a rapid monomer-dimer equilibrium. (BSA control behaved as expected validating the DAT0120 experimental results, giving two peaks with molar mass of 61 kDa and 146 kDa (monomer and dimer)). See FIG. 8 for SEC Mal results.
It was concluded from these experiments above that DAT0115 demonstrates significantly less (and possibly no) self association under the conditions used here compared to the other two molecules which show significant elf association. In-solution monomeric state may be preferable with regards to in vivo action and upstream and downstream process during manufacturing so DAT0115 may be the most ideal molecule for clinical progression with regards in-solution state.

### Example 15

### Purification from Mammalian Expression without Using the Affinity Matrix Protein L

Both DAT0120 and DAT0115 were purified from HEK 293 supernatants. Each protein was expressed in mammalian tissue culture in HEK 293E cells from the pTT-5 vector. A 1 ml column of MEP Hypercel resin was equilibrated with PBS, washed with 0.1M Sodium Hydroxide and then re-equilibrated with PBS. 200 ml of supernatant was applied to the column at 2.5 ml/min and then the column was washed with PBS and eluted with 0.1M Glycine, pH2.
Post elution the sample was neutralised by addition of ⅕^{th} volume of 1M Tris, pH 8 and stored at room temperature. The sample showed light precipitation after storage and was filtered using a steriflip device prior to desalting.
Two 26/10 HiPrep desalting columns were equilibrated with 20 mM Sodium Acetate, pH5 (measured pH 5.3) at 10 ml/min, cleaned by addition of 0.1M NaOH and re-equilibrated into 20 mM Sodium Acetate, pH5.
DAT0115 was desalted into 20 mM Sodium Acetate, pH5 prior to loading on 1 ml HiTrap SPFF equilibrated in 20 mM Sodium Acetate, pH5 (actual 5.2). Post washing of the column it was subjected to a 0-100% gradient with 20 mM Sodium Acetate, pH5, 1M NaCl and elution fractions with absorbance over 5 mAus were collected and analysed by SDS-PAGE.
Post storage of the SP FF fractions overnight the sample was 0.2 um filtered and applied to 2×26/10 HiPrep desalting columns equilibrated into 20 mM Sodium Citrate, pH6.2, 100 mM NaCl. The elution was concentrated in a 20 ml centrifugal concentrator, filter sterilised and endotoxin tested at dilutions 1/10 and 1/200.
Endotoxin was tested at two dilutions, the 1/10 dilution gave a value of 30 Eu/ml with a spike recovery of 250. The 1/200 test gave a value of <10.8 Eu/ml with a spike recovery of 126%. The sample was submitted for MS analysis using the ID 27823. There are low level contaminants visible below the 80 kDa marker and between the 110-160 kDa markers in the high loading. The sample looks to be greater than 95% pure.

1. A fusion or conjugate composition comprising (a) an insulinotropic agent or an incretin drug present as a fusion or a conjugate with, (b) the DOM 7h-14 domain antibody (dAb) which binds serum albumin and comprises the amino acid sequence of SEQ ID NO 8.

2. The fusion or conjugate according to claim 1, wherein the drug is an exendin-4, or a GLP-1 molecule.

3. The fusion or conjugate according to claim 1, wherein the drug is selected from (a) the GLP-1 (7-37) A8G mutant comprising the amino acid sequence of SEQ ID NO 9, or (b) the exendin-4 molecule comprising the amino acid sequence of SEQ ID NO 10.

4. The fusion or conjugate according to claim 1, which comprises an amino acid or chemical linker joining the drug and the dAb.

5. The fusion or conjugate according to claim 4, wherein the amino acid linker is a helical linker comprising the amino acid sequence of SEQ ID NO 11, or the gly-ser linker comprising the amino acid sequence of SEQ ID NO 12.

6. The fusion according to claim 1, wherein the insulinotropic agent or the incretin drug is present as part of a fusion at either the N-terminal or C-terminal of the dAb.

7. The fusion according to claim 6, which comprises or consists of an amino acid sequence selected from the group consisting of:
(a) 2×GLP-1 A8G DOM7h-14 fusion (DAT0114)
(SEQ ID NO 1)
HGEGTFTSDVSSYLEGQAAKEFIAWLVKGRHGEGTFTSDVSSYLEG
QAAKEFIAWLVKGRDIQMTQSPSSLSASVGDRVTITCRASQWIGSQ
LSWYQQKPGKAPKLLIMWRSSLQSGVPSRFSGSGSGTDFTLTISSL
QPEDFATYYCAQGAALPRTFGQGTKVEIKR
(b) Exendin 4, (G4S)3 linker, DOM7h-14 fusion (DAT0115)
(SEQ ID NO 2)
HGEGTFTSDLSKQMEEEAVRLFIEWLKNGGPSSGAPPPSGGGGGSG
GGGSGGGGSDIQMTQSPSSLSASVGDRVTITCRASQWIGSQLSWYQ
QKPGKAPKLLIMWRSSLQSGVPSRFSGSGSGTDFTLTISSLQPEDF
ATYYCAQGAALPRTFGQGTKVEIKR
(c) Exendin 4 DOM7h-14 fusion (DAT0116)
(SEQ ID NO 3)
HGEGTFTSDLSKQMEEEAVRLFIEWLKNGGPSSGAPPPSGDIQMTQS
PSSLSASVGDRVTITCRASQWIGSQLSWYQQKPGKAPKLLIMWRSSL
QSGVPSRFSGSGSGTDFTLTISSLQPEDFATYYCAQGAALPRTFGQG
TKVEIKR
(d) Exendin 4, helical linker, DOM7h-14 fusion (DAT0117)
(SEQ ID NO 4)
HGEGTFTSDLSKQMEEEAVRLFIEWLKNGGPSSGAPPPSGKEAAAKE
AAAKEAAAKELAAKEAAAKEAAAKEAAAKELAADIQMTQSPSSLSAS
VGDRVTITCRASQWIGSQLSWYQQKPGKAPKLLIMWRSSLQSGVPSR
FSGSGSGTDFTLTISSLQPEDFATYYCAQGAALPRTFGQGTKVEIKR
(e) GLP-1 A8G, (G4S)3, linker DOM7h-14 fusion (DAT0118)
(SEQ ID NO 5)
HGEGTFTSDVSSYLEGQAAKEFIAWLVKGRGGGGSGGGGSGGGGSDI
QMTQSPSSLSASVGDRVTITCRASQWIGSQLSWYQQKPGKAPKLLIM
WRSSLQSGVPSRFSGSGSGTDFTLTISSLQPEDFATYYCAQGAALPR
TFGQGTKVEIKR
(f) GLP-1 A8G, PSS linker, DOM7h-14 fusion (DAT0119)
(SEQ ID NO 6)
HGEGTFTSDVSSYLEGQAAKEFIAWLVKGRGPSSDIQMTQSPSSLSAS
VGDRVTITCRASQWIGSQLSWYQQKPGKAPKLLIMWRSSLQSGVPSRF
SGSGSGTDFTLTISSLQPEDFATYYCAQGAALPRTFGQGTKVEIKR;
and
(g) GLP-1 A8G, helical linker, DOM7h-14 fusion (DAT0120)
(SEQ ID NO 7)
HGEGTFTSDVSSYLEGQAAKEFIAWLVKGRGKEAAAKEAAAKEAAA
KELAAKEAAAKEAAAKEAAAKELAADIQMTQSPSSLSASVGDRVTI
TCRASQWIGSQLSWYQQKPGKAPKLLIMWRSSLQSGVPSRFSGSGS
GTDFTLTISSLQPEDFATYYCAQGAALPRTFGQGTKVEIKR.

8. The fusion or conjugate according to claim 1, wherein the dAb is further formatted to increase its hydrodynamic size by attaching at least one molecules to the dAb selected from the group consisting of: a PEG group, serum albumin, transferrin, transferrin receptor or at least the transferrin-binding portion thereof, an antibody Fc region, by conjugation to an antibody domain, and combinations thereof.

9. The fusion or conjugate according to claim 1, which comprises a further peptide or polypeptide moiety.

10. The fusion or conjugate according to claim 1, which comprises additional dAb moieties which have the same or different binding specificities to the Dom7h-14 dAb.

11. The fusion or conjugate according to claim 1 which has an elimination half life in a human of at least 12 hours.

12. The fusion or conjugate according to claim 1 which binds to human serum albumin with KD in the range of about 5 micromolar to about 1 picomolar.

13. A pharmaceutical composition comprising a fusion or conjugate according to claim 1 in combination with a pharmaceutically or physiologically acceptable carrier, excipient or diluent.

14. A pharmaceutical composition according to claim 13, which comprises further therapeutic or active agents.

15. A composition which comprises a (a) fusion or conjugate according to claim 1 and (b) further therapeutic or active agents, for separate, sequential or concurrent administration to a subject.

16. The composition according to claim 1, for use in treating or preventing a metabolic disease or disorder.

17. The composition according to claim 16, wherein the disease or disorder is at least one selected from the group consisting of: hyperglycemia, impaired glucose tolerance, beta cell deficiency, diabetes (type 1 or type 2 diabetes or gestational diabetes), obesity, and diseases characterised by overeating.

18. A method of treating or preventing a metabolic disease comprising administering to a patient a therapeutically or prophylactically effective amount of a composition according to claim 1.

19. An oral, injectable, inhalable or nebulisable formulation which comprises a composition according to claim 1.

20. A sustained release formulation e.g. in the form of a suppository which comprises a composition according to claim 1.

21. A freeze dried formulation which comprises a composition according to claim 1.

22. A delivery device comprising a composition according to claim 1.

23. An isolated or recombinant nucleic acid encoding a fusion according to claim 1.

24. A nucleic acid encoding the fusions of claim 7.

25. A vector comprising a nucleic acid of claim 23.

26. A host cell comprising the nucleic acid of claim 23.

27. A method of producing a fusion polypeptide comprising (a) an insulinotropic agent or an incretin drug present as a fusion with, (b) the DOM 7h-14 domain antibody (dAb) which binds serum albumin and which has the amino acid sequence shown in FIG. 1(h), the method comprising maintaining a host cell of claim 26 under conditions suitable for expression of said nucleic acid or vector, whereby a fusion polypeptide is produced.

28. A method of treating or preventing a disease or disorder associated with elevated blood glucose in a patient e.g. a human patient, comprising administering to said patient a therapeutically or prophylactically effective amount of a composition according to claim 1.

29. A method of stimulating insulin production and/or increasing insulin sensitivity in a patient e.g. a human patient, comprising administering to said patient at least one dose of a composition according to claim 1.

30. The method of claim 18, wherein the composition is administered to the subject by subcutaneous, intravenous or intramuscular injection.

31. The method of claim 18, wherein the composition is administered to the subject by parenteral, oral, rectal, transmucosal, ocular, pulmonary or GI tract delivery.
